(19) United States
(12) Patent Application Publication
(10) Pub. No.: US 2007/0037776 A1
(43) Pub. Date: Feb. 15, 2007

(54) Polysaccharides for delivery of active agents

(76) Inventors: Tom Richardson, Princeton Junction, NJ (US); Chia Lin Chu, Somerville, MA (US); Jeff Hrkach, Lexington, MA (US)

Correspondence Address: FISH & RICHARDSON PC, P.O. BOX 1022, MINNEAPOLIS, MN, 55440-1022, US

(21) Appl. No.: 11/500,612

(22) Filed: Aug. 8, 2006

Related U.S. Application Data
(60) Provisional application No. 60/706,320, filed on Aug. 8, 2005.

(51) Int. Cl. A61K 31/715

(52) U.S. Cl. 514/54; 514/61

(57) ABSTRACT

Formulation and methods for modulating the delivery of an agent using polysaccharides.

### CLAIM OF PRIORITY

This application claims priority under 35 USC §119(e) to U.S. Patent Application Ser. No. 60/706,320, filed on Aug. 8, 2005, the entire contents of each of which is hereby incorporated by reference.

### BACKGROUND OF THE INVENTION

Epithelial cells provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. The tight junction (TJ) of epithelial and endothelial cells is a particularly important cell-cell junction that regulates permeability of the paracellular pathway, and also divides the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration which occurs primarily along the paracellular rout and appears to be regulated via opening and closing of tight junctions in a highly coordinated and reversible manner.

Numerous proteins have been identified in association with tight junctions, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other tight junction-associated molecules designated as occludins, claudins, and zonulin.

### SUMMARY OF THE INVENTION

The present invention is based, in part, on the discovery that various properties of polysaccharides (and non-polysaccharide polymers that have similar properties to these polysaccharides) modulate movement of active agents through epithelial tissue. Thus, polysaccharides (and non-polysaccharide polymers) having these properties allow for enhanced delivery of active agents across epithelial barriers as compared to delivery of the agent in the absence of the polysaccharide or non-polysaccharide polymer, e.g., the polysaccharide or non-polysaccharide polymer allows for delivery of the agent at therapeutically and prophylactically effective levels. In addition, it has been found that other properties of polysaccharides (and non-polysaccharide polymers) reduce or impede movement of agents across epithelial barriers by altering tight junctions. Properties that have been found to effect migration include the level of sulfation, e.g., N-sulfation or 2-O sulfation, molecular weight and activity level of the polysaccharide.

Accordingly, in one aspect, the invention features a method of modulating, e.g., increasing or decreasing, movement of an agent across epithelium. The method includes: contacting epithelial tissue with a polysaccharide, e.g., a soluble polysaccharide, capable of altering, e.g., increasing or decreasing the permeability of, intercellular junctions between epithelial cells; and contacting epithelial tissue with an agent, to thereby modulate movement of the agent through the epithelial tissue.

In one embodiment, the polysaccharide is a hexasaccharide or larger polysaccharide, e.g., an octasaccharide, decasaccharide.

In one embodiment, the polysaccharide is contacted with the epithelial tissue prior to contacting the epithelial tissue with the agent, e.g., the agent is contacted with the epithelial tissue while the modulating effect of the polysaccharide is still active.

In another embodiment, the agent is contacted with the epithelial tissue prior to contacting the epithelial tissue with the polysaccharide, e.g., the polysaccharide is contacted with the epithelial tissue while an effect of the agent is still present, e.g., the polysaccharide is contacted with the epithelial tissue such that movement of the agent across an epithelium is increased or decreased as compared to contacting the epithelial tissue with the agent in the absence of the polysaccharide. In another embodiment, the agent is contacted with the epithelial tissue simultaneously with the polysaccharide.

In one embodiment, the epithelial tissue includes pulmonary epithelial cells.

In another embodiment, the epithelial tissue includes ocular epithelial cells, dermal epithelial cells, vaginal epithelial cells, nasal epithelial cells, or epithelial cells in the mouth and/or throat.

In one embodiment, the polysaccharide is capable of modulating movement of the agent across an epithelium upon contact or within 1, 5, 10, 15, 20, 25, 30 minutes after contact with the polysaccharide. In one embodiment, the polysaccharide is capable of modulating movement of the agent across an epithelium for a period of about 30 minutes to 5 hours, about 1 hour to 4 hours, about 2 hours to 3 hours.

In one embodiment, the polysaccharide is a polysaccharide capable of increasing movement of the agent across an epithelium, e.g., an agent described herein, e.g., a therapeutic, prophylactic or diagnostic agent. The polysaccharide can be, e.g., a polysaccharide described herein. The polysaccharide can be contacted with the epithelial tissue prior to, simultaneously, or after the epithelial tissue has been contacted with the agent. For example, the polysaccharide can be contacted with the epithelial tissue prior to contacting the epithelial tissue with the agent (e.g., the agent is contacted with the epithelial tissue while the ability of the polysaccharide to increase movement is still present, e.g., such that movement of the agent is increased as compared to contacting the tissue with the agent in the absence of the polysaccharide); the agent can be contacted with the epithelial tissue prior to contacting the epithelial tissue with the polysaccharide (e.g., the polysaccharide is contacted with the epithelial tissue while an effect of the agent is still detectable, e.g., such that the effect of the agent is increased as compared to contacting the tissue with the agent in the absence of the polysaccharide).

In another embodiment, the agent is contacted with the epithelial tissue simultaneously with the polysaccharide, e.g., such that the movement of the agent across an epithelium is increased as compared to contacting the epithelial tissue with the agent in the absence of the polysaccharide.

In one embodiment, when movement across an epithelium is increased, the polysaccharide is an HLGAG, e.g., a heparin or LMWH, e.g., a LMWH selected from enoxaparin, dalteparin, reviparin, tinzaparin, nadroparin, certoparin, ardeparin, M118 and parnaparin.

In one embodiment, the polysaccharide is a polysaccharide capable of decreasing movement of the agent across an epithelium, e.g., an agent described herein, e.g., a pathogenic agent, e.g., a virion, an allergen or a bacteria. The polysaccharide can be, e.g., a polysaccharide described herein. The polysaccharide can be contacted with the epithelial tissue prior to, simultaneously, or after the epithelial tissue has been contacted with the agent. For example, the polysaccharide can be contacted with the epithelial tissue prior to contact of the epithelial tissue with the agent (e.g., the agent is contacted with the epithelial tissue while the ability of the polysaccharide to decrease movement across an epithelium is still present, e.g., such that movement of the agent across an epithelium is decreased as compared to contacting the tissue with the agent in the absence of the polysaccharide); or the agent can be contacted with the epithelial tissue simultaneously with the polysaccharide, e.g., such that the movement of the agent across an epithelium is decreased as compared to contacting the epithelial tissue with the agent in the absence of the polysaccharide.

In one embodiment, when movement across an epithelium is decreased, the polysaccharide is a chondroitin sulfate, a dermatan sulfate, or a 2-O-desulfated heparin.

In one aspect, the invention features a method of modulating, e.g., increasing or decreasing, movement of an agent across an epithelium. The method includes: contacting epithelial tissue with a polysaccharide, e.g., a soluble polysaccharide, capable of modulating one or more of the location, expression level and activity of one or more intercellular junction proteins; and contacting epithelial tissue with an agent, to thereby modulate movement of the agent through the epithelial tissue. In one embodiment, the intercellular junction protein is selected from a claudin, a junction associate molecule (JAM), an occludin, and zona occludin, e.g., a zonula occluden, zona occludin-1 (ZO-1), zona occluding-2 (ZO-2) and/or zona occulin-3 (ZO-3). Preferably, the polysaccharide is capable of altering, e.g.,increasing, the expression and/or activity of ZO-1.

In one embodiment, the polysaccharide is a hexasaccharide or larger polysaccharide, e.g., an octasaccharide, decasaccharide.

In one embodiment, the polysaccharide is contacted with the epithelial tissue prior to contacting the epithelial tissue with the agent, e.g., the agent is contacted with the epithelial tissue while the modulating effect of the polysaccharide is still detectable.

In another embodiment, the agent is contacted with the epithelial tissue prior to contacting the epithelial tissue with the polysaccharide, e.g., the polysaccharide is contacted with the epithelial tissue while an effect of the agent is still detectable, e.g., the polysaccharide is contacted with the epithelial tissue such that movement of the agent across an epithelium is increased or decreased as compared to contacting the epithelial tissue with the agent in the absence of the polysaccharide. In another embodiment, the agent is contacted with the epithelial tissue simultaneously with the polysaccharide.

In one embodiment, the epithelial tissue includes pulmonary epithelial cells.

In another embodiment, the epithelial tissue includes ocular epithelial cells, dermal epithelial cells, or nasal epithelial cells.

In one embodiment, the polysaccharide is capable of modulating movement of the agent across an epithelium upon contact or within 1, 5, 10, 15, 20, 25, 30 minutes after contact with the polysaccharide. In one embodiment, the polysaccharide is capable of modulating movement of the agent across an epithelium for a period of about 30 minutes to 5 hours, about 1 hour to 4 hours, about 2 hours to 3 hours.

In one embodiment, the polysaccharide is a polysaccharide capable of increasing movement of the agent across an epithelium, e.g., an agent described herein, e.g., a therapeutic, prophylactic or diagnostic agent. The polysaccharide can be, e.g., a polysaccharide described herein. The polysaccharide can be contacted with the epithelial tissue prior to, simultaneously, or after the epithelial tissue has been contacted with the agent. For example, the polysaccharide can be contacted with the epithelial tissue prior to contacting the epithelial tissue with the agent (e.g., the agent is contacted with the epithelial tissue while the ability of the polysaccharide to increase movement across an epithelium is still present, e.g., such that movement of the agent across an epithelium is increased as compared to contacting the tissue with the agent in the absence of the polysaccharide); the agent can be contacted with the epithelial tissue prior to contacting the epithelial tissue with the polysaccharide (e.g., the polysaccharide is contacted with the epithelial tissue while an effect of the agent is still detectable, e.g., such that the effect of the agent is increased as compared to contacting the tissue with the agent in the absence of the polysaccharide). In another embodiment, the agent is contacted with the epithelial tissue simultaneously with the polysaccharide, e.g., such that the movement of the agent across an epithelium is increased as compared to contacting the epithelial tissue with the agent in the absence of the polysaccharide.

In one embodiment, when movement across an epithelium is increased, the polysaccharide is a polysaccharide described herein such as an HLGAG, e.g., a heparin or LMWH, e.g., a LMWH selected from enoxaparin, dalteparin, reviparin, tinzaparin, nadroparin, certoparin, ardeparin, M118 and parnaparin.

In one embodiment, the polysaccharide is a polysaccharide capable of decreasing movement of the agent across an epithelium, e.g., an agent described herein, e.g., a pathogenic agent, e.g., a virion, an allergen or a bacteria. The polysaccharide can be contacted with the epithelial tissue prior to, simultaneously, or after the epithelial tissue has been contacted with the agent. For example, the polysaccharide can be contacted with the epithelial tissue prior to contact of the epithelial tissue with the agent (e.g., the agent is contacted with the epithelial tissue while the ability of the polysaccharide to decrease movement of the agent across an epithelium is still present, e.g., such that movement of the agent across an epithelium is decreased as compared to contacting the tissue with the agent in the absence of the polysaccharide); or the agent can be contacted with the epithelial tissue simultaneously with the polysaccharide, e.g., such that the movement of the agent across an epithelium is decreased as compared to contacting the epithelial tissue with the agent in the absence of the polysaccharide.

In one embodiment, when movement of the agent across an epithelium movement is decreased, the polysaccharide is a polysaccharide described herein such as, e.g., chondroitin sulfate, a dermatan sulfate, or a 2-O-desulfated heparin.

In another aspect, the invention features a method for decreasing the permeability of intercellular junctions, e.g., tight junctions, in epithelial tissue. The method includes: contacting the epithelial tissue with a polysaccharide, e.g., a soluble polysaccharide, to thereby decrease the permeability of the intercellular junctions. Preferably, the epithelial tissue is contacted with an amount of polysaccharide to alter expression and/or activity of a zona occludin, e.g., a zonula occluden, zona occludin-1 (ZO-1), zona occludin-2 (ZO-2) and/or zona occulin-3 (ZO-3). In one embodiment, the polysaccharide reduces the ability of an agent to penetrate the epithelial tissue.

In one embodiment, the polysaccharide is a hexasaccharide or larger polysaccharide, e.g., an octasaccharide, decasaccharide or larger.

In one embodiment, the polysaccharide is a polysaccharide described herein as a polysaccharide that reduces the permeability through intercellular junctions. In one embodiment, the polysaccharide is: a chondroitin sulfate, a dermatan sulfate, or an HLGAG that has no 2-O sulfate groups or a polysaccharide that is less than 40%, 30%, 20%, 10%, 5% 2-O-sulfated. In one embodiment, when the polysaccharide is an HLGAG, the HLGAG has been modified to decrease 2-O-sulfation, e.g., the HLGAG has been chemically or enzymatically treated to decrease 2-O sulfation of the polysaccharide, by at least 5%, 10%, 20%, 30%, 40%, 50%, 60%, 70%, 80%, 90%, 95% or more. For example, the HLGAG can be enzymatically treated with a 2-O sulfatase. Alternatively, the HLGAG can be 2-O desulfated with chemical methods known in the art, e.g., refluxing in sodium carbonate or treatment with methanol/DMSO solution for a period of time sufficient to remove 2-O sulfates.

In one embodiment, the agent is an agent described herein such as a pathogenic molecule, 5e.g., a pathogenic molecule selected from virions, allergens and bacteria. In one embodiment, the pathogenic molecule is a pathogenic biowarfare molecule, e.g., anthrax, ricin, brucellosis, cholera, Congo-Crimean hemorrhagic fever, ebola hemorrhagic fever, Marburg fever, melioidosis, plague, Q fever, rift valley fever, saxitoxin, smallpox, staphylococcal enterotoxin B, tricothecene mycotoxins, tularemia, Venezuelan equine encephalitis and botulinum toxin. In one embodiment, the pathogenic molecule is selected from the group consisting of: Clostridium perfringens, Clostridium diphtheriae, Clostridium difficile, Vibrio cholerae, Escherichia coli, bacterioides fragilis, Helicobacter pylori, Dermatophagoides pteronyssinus, reovirus, Coxsackievirus, and rotavirus. In one embodiment, the pathogenic molecule is a virus selected from the group consisting of: HSV, HPV, RSV, HIV, and AAV.

In one embodiment, the polysaccharide enhances the permeability of intercellular junctions upon contact or within 1, 5, 10, 15, 20, 25, 30 minutes after contact. In another embodiment, the polysaccharide decreases the permeability of the intercellular junctions for a period of about 30 minutes to 5 hours, about 1 hour to 4 hours, about 2 hours to 3 hours. In one embodiment, the polysaccharide is contacted with the epithelial tissue prior to contacting the epithelial tissue with the agent, e.g., the agent is contacted with the epithelial tissue while the modulating effect of the polysaccharide is still detectable.

In another embodiment, the agent is contacted with the epithelial tissue prior to contacting the epithelial tissue with the polysaccharide, e.g., the polysaccharide is contacted with the epithelial tissue while an effect of the agent is still detectable, e.g., the polysaccharide is contacted with the epithelial tissue such that movement of the agent across an epithelium is increased or decreased as compared to contacting the epithelial tissue with the agent in the absence of the polysaccharide. In another embodiment, the agent is contacted with the epithelial tissue simultaneously with the polysaccharide.

In one embodiment, the epithelial tissue includes pulmonary epithelial cells.

In another embodiment, the epithelial tissue includes ocular epithelial cells, dermal epithelial cells, nasal epithelial cells, vaginal epithelial cells or epithelial cells in the mouth and/or throat.

In another aspect, the invention features a method for non-mucosal delivery of an effective amount of an agent, e.g., an agent described herein, e.g., a therapeutic, prophylactic or diagnostic agent, to a subject. The method includes: administering to a subject an effective amount of the agent and a polysaccharide, e.g., a soluble polysaccharide, to thereby deliver the agent to the subject. The non-mucosal delivery can be, e.g., transdermal delivery.

In one embodiment, the polysaccharide is a hexasaccharide or larger polysaccharide, e.g., an octasaccharide, decasaccharide or larger.

In one embodiment, the agent can be a therapeutic or prophylactic polypeptide, nucleic acid, small molecule, lipid/glycolipids, etc. In one embodiment, the active agent is a therapeutic polypeptide selected from the group consisting of insulin, proinsulin, human growth hormone, interferon, α-1 proteinase inhibitor, alkaline phosphatase, angiogenin, cystic fibrosis transmembrane conductance regulator, extracellular superoxide dismutase, fibrinogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin. The therapeutic or prophylactic polypeptide can be an active derivative or fragment of such polypeptides. The active agent can also be, but is not limited to one or more of: parathyroid hormone and derivatives and fragments thereof, erythropoietin, epoetin beta, gene activated erythropoietin, second generation EPO, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, domase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/ somatropin, anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, sargramostim, lenograstim, molgramostim, mirimostim, nartograstim, oprelvekin, peptide tyrosin-tyrosin (PYY), apolipoprotein A-IV, leptin, melanocortin, amylin, orexin, adiponectin, and ghrelin. In one embodiment, the active agent is an active polypeptide, e.g., a therapeutic or prophylactic polypeptide, and the polypeptide has a molecular weight of about 150 kDa or less, about 100 kDa or less, about 50 kDa or less, or about 0.5-35 kDa or less. In one embodiment, the active agent is an active polypeptide, e.g., a therapeutic or prophylactic polypeptide, and the polypeptide has a molecular weight of about: 500 Da-5 kDa, 5 to 10 kDa, 10 to 30 kDa, 18 to 35 kDa, 30 to 50 kDa, 50 to 100 kDa, 100 to 150 kDa. In one embodiment, the active polypeptide is insulin or an active fragments or derivatives thereof. In another embodiment, the active polypeptide is human growth hormone or an active fragment or derivative thereof. In yet another embodiment, the active polypeptide is interferon.

In other embodiments, the agent is an inactive agent. Examples of inactive agents include biological probes or contrast agents for imaging.

In another embodiment, the agent can be a small molecule drug, e.g., a small molecule drug currently available for therapeutic, diagnostic, or prophylactic use, or a drug in development. In some embodiments, the agent can be admixed with the polysaccharide. Admixtures can be prepared, e.g., by mixing, covalently-linked polysaccharides, ionically-linked polysaccharides, spraying drying and other techniques known in the art. In some embodiments, the agent is linked to one or more polysaccharides in the formulation. As an example, small molecule drugs, and protein-based drugs may be linked to polysaccharides for delivery via known chemistries such as EDC, CNBH4/DMSO/Acetic Acid, etc.

In one embodiment, the polysaccharide is capable of increasing delivery of the agent upon administration or within 1, 5,10, 15, 20, 25, 30 minutes after administration of the polysaccharide. In one embodiment, the polysaccharide is capable of increasing delivery of the agent for a period of about 30 minutes to 5 hours, about 1 hour to 4 hours, about 2 hours to 3 hours after administration, e.g., as compared to delivery of the agent in the absence of the polysaccharide. In one embodiment, the polysaccharide is administered prior to administration of the agent, e.g., the agent is administered while the modulating effect of the polysaccharide is still detectable. In another embodiment, the agent is administered prior to administration of the polysaccharide, e.g., the polysaccharide is administered while an effect of the agent is still detectable, e.g., the polysaccharide is administered such that delivery of the agent is increased as compared to contacting the epithelial tissue with the agent in the absence of the polysaccharide. In another embodiment, the agent is administered simultaneously with the polysaccharide.

In some embodiments, the polysaccharide and the agent are in a composition, e.g., a composition further comprising a pharmaceutically acceptable carrier and/or a delivery enhancer.

In one embodiment, the polysaccharide is in a preparation of polysaccharides, e.g., a preparation of polysaccharides comprising LMWH, e.g., a preparation comprising LMWH wherein all or a portion of the polysaccharides in the preparation consist of about two to twenty monosaccharides. In one embodiment, the polysaccharide is an HLGAG, e.g., heparin or a LMWH, e.g., a LMWH selected from enoxaparin, dalteparin, reviparin, tinzaparin, nadroparin, certoparin, ardeparin, M118 and parnaparin.

In one embodiment, the polysaccharide is a heparin or LMWH that has been modified to alter one or more of its charge, size, level of sulfation or therapeutic activity, e.g., by a method described herein. In one embodiment, the chemical signature of one or more polysaccharides in the preparation has been determined and, e.g., one or more polysaccharide is modified based upon its chemical signature.

In one embodiment, the polysaccharide is a heparin or LMWH that has an anti-Xa activity and/or an anti-IIa activity that is reduced by at least 10%, 20%, 30%, 40%, 50%, 60%, 70% 80%, 90% or more as compared to a reference standard and, e.g., the reference standard is the level of anti-Xa activity and/or anti-IIa activity of a commercially available version of the heparin or LMWH or is the level of anti-Xa activity and/or anti-IIa activity of the heparin or LMWH prior to modification. In some embodiments, the heparin or LMWH is modified at one or more chemical signature of an oligosaccharide of heparin which comprises the structure ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. to reduce the anti-Xa activity and/or the anti-IIa activity of the heparin or LMWH. In some embodiments, the heparin or LMWH further has PF4 binding and/or FGF-2 binding that is reduced by at least 10%, 20%, 30%, 40%, 50%, 60%, 70% or more than a reference standard. The heparin or LMWH can be, e.g., a heparin described herein.

In some embodiments, the heparin or LMWH comprises a size that is reduced as compared to a reference standard, e.g., the reference standard is the level of average chain length of a commercially available version of the heparin or LMWH or is the average chain length of the heparin or LMWH prior to modification. In some embodiments, the heparin or LMWH is a heparin or LMWH with a reduced size is a heparin or LMWH described herein, e.g., a heparin or LMWH that has been modified to reduce its size by a method described herein.

In some embodiments, the heparin or LMWH comprises a charge that has been modified as compared to a reference standard, e.g., the reference standard is the charge of a commercially available version of the heparin or LMWH or is the charge of the heparin or LMWH prior to modification. In some embodiments, the heparin or LMWH is a heparin or LMWH with a modified charge is a heparin or LMWH described herein, e.g., a heparin or LMWH having a charge that has been modified by a method described herein.

In some embodiments, the polysaccharide has no N-sulfate groups or the polysaccharide that is less than 40%, 30%, 20%, 10% N-sulfated. In some embodiments, the polysaccharide is a heparin or LMWH that has been modified to decrease N-sulfation, e.g., the heparin or LMWH has been chemical or enzymatically treated to decrease N-sulfation of the heparin or LMWH, by at least 5%, 10%, 20%, 30%, 40%, 50%, 60%, 70%, 80%, 90%, 95% or more. In some embodiments, the heparin or LMWH is a heparin or LMWH is a heparin or LMWH described herein, e.g., a heparin or LMWH having a level of sulfation that has been modified by a method described herein.

In one aspect, the invention features a method for ocular delivery of an effective amount of an agent that includes administering to a subject an effective amount of an agent and a polysaccharide.

In one embodiment, the polysaccharide is a hexasaccharide or larger polysaccharide, e.g., an octasaccharide, decasaccharide or larger.

In one embodiment, the agent can be a therapeutic or prophylactic polypeptide, nucleic acid, small molecule, lipid/glycolipids, etc. In one embodiment, the active agent is a therapeutic polypeptide selected from the group consisting of insulin, proinsulin, human growth hormone, interferon, α-1 proteinase inhibitor, alkaline phosphotase, angiogenin, cystic fibrosis transmembrane conductance regulator, extracellular superoxide dismutase, fibrinogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin. The therapeutic or prophylactic polypeptide can be an active derivative or fragment of such polypeptides. The active agent can also be, but is not limited to one or more of: parathyroid hormone and derivatives and fragments thereof, erythropoietin, epoetin beta, gene activated erythropoietin, second generation EPO, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, dornase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/somatropin, anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, sargramostim, lenograstim, molgramostim, mirimostim, nartograstim, oprelvekin, peptide tyrosin-tyrosin (PYY), apolipoprotein A-IV, leptin, melanocortin, amylin, orexin, adiponectin, and ghrelin. In one embodiment, the active agent is an active polypeptide, e.g., a therapeutic or prophylactic polypeptide, and the polypeptide has a molecular weight of about 150 kDa or less, about 100 kDa or less, about 50 kDa or less, or about 0.5-35 kDa or less. In one embodiment, the active agent is an active polypeptide, e.g., a therapeutic or prophylactic polypeptide, and the polypeptide has a molecular weight of about: 500 Da-5 kDa, 5 to 10 kDa, 10 to 30 kDa, 18 to 35 kDa, 30 to 50 kDa, 50 to 100 kDa, 100 to 150 kDa. In one embodiment, the active polypeptide is insulin or an active fragments or derivatives thereof. In another embodiment, the active polypeptide is human growth hormone or an active fragment or derivative thereof. In yet another embodiment, the active polypeptide is interferon.

In other embodiments, the agent is an inactive agent. Examples of inactive agents include biological probes or contrast agents for imaging.

In another embodiment, the agent can be a small molecule drug, e.g., a small molecule drug currently available for therapeutic, diagnostic, or prophylactic use, or a drug in development. In some embodiments, the agent is admixed with the polysaccharide. Admixtures can be prepared, e.g., by mixing, spray drying and other techniques known in the art. In some embodiments, the agent is linked to one or more polysaccharides in the formulation. As an example, small molecule drugs, and protein-based drugs may be linked to polysaccharides for delivery via known chemistries such as EDC, CNBH4/DMSO/Acetic Acid, etc.

In one embodiment, the polysaccharide is capable of modulating movement of the agent across an epithelium into the eye upon contact or within 1, 5, 10, 15, 20, 25, 30 minutes after contact with the polysaccharide. In one embodiment, the polysaccharide is capable of modulating movement of the agent across an epithelium into the eye for a period of about 30 minutes to 5 hours, about 1 hour to 4 hours, about 2 hours to 3 hours. In one embodiment, the polysaccharide is administered prior to administration of the agent, e.g., the agent is administered while the modulating effect of the polysaccharide is still detectable. In another embodiment, the agent is administered prior to administration of the polysaccharide, e.g., the polysaccharide is administered while an effect of the agent is still detectable, e.g., the polysaccharide is administered such that delivery of the agent is increased as compared to contacting the epithelial tissue with the agent in the absence of the polysaccharide. In another embodiment, the agent is contacted with the epithelial tissue simultaneously with the polysaccharide.

In some embodiments, the polysaccharide and the agent are in a composition, e.g., a composition further comprising a pharmaceutically acceptable carrier and/or a delivery enhancer.

In one embodiment, the polysaccharide is in a preparation of polysaccharides, e.g., a preparation of polysaccharides comprising LMWH, e.g., a preparation comprising LMWH wherein all or a portion of the polysaccharides in the preparation consist of about two to twenty monosaccharides. In one embodiment, the polysaccharide is an HLGAG, e.g., heparin or a LMWH, e.g., a LMWH selected from enoxaparin, dalteparin, reviparin, tinzaparin, nadroparin, certoparin, ardeparin, M118 and parnaparin.

In one embodiment, the polysaccharide is a heparin or LMWH that has been modified to alter one or more of its charge, size, level of sulfation or therapeutic activity, e.g., by a method described herein. In one embodiment, the chemical signature of one or more polysaccharides in the preparation has been determined and, e.g., one or more polysaccharide is modified based upon its chemical signature.

In one embodiment, the polysaccharide is a heparin or LMWH that has an anti-Xa activity and/or an anti-IIa activity that is reduced by at least 10%, 20%, 30%, 40%, 50%, 60%, 70% 80%, 90% or more as compared to a reference standard and, e.g., the reference standard is the level of anti-Xa activity and/or anti-IIa activity of a commercially available version of the heparin or LMWH or is the level of anti-Xa activity and/or anti-IIa activity of the heparin or LMWH prior to modification. In some embodiments, the heparin or LMWH is modified at one or more chemical signature of an oligosaccharide of heparin which comprises the structure ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. to reduce the anti-Xa activity and/or the anti-IIa activity of the heparin or LMWH. In some embodiments, the heparin or LMWH further has PF4 binding and/or FGF-2 binding that is reduced by at least 10%, 20%, 30%, 40%, 50%, 60%, 70% or more than a reference standard.

In one embodiment, the polysaccharide is a heparin or LMWH having a size that is reduced as compared to a reference standard, e.g., the reference standard is the level of average chain length of a commercially available version of the heparin or LMWH or is the average chain length of the heparin or LMWH prior to modification. In one embodiment, the mass of the polysaccharide is reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided polysaccharide. In other embodiments, the mass of the polysaccharide is reduced by at least 5, 10, 15, 20, 25, 30, 35, 40, 50, 60, 70, 80, 90, 100,150, 200, 250, 300, 350, 500, 1000, 1500, 2000, 2200, 2500, 3000 Da or more from the mass of the provided polysaccharide. In one embodiment, the mass of the provided polysaccharide can be reduced, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In one embodiment, the polysaccharide is a heparin or LMWH having a charge that has been modified as compared to a reference standard, e.g., the reference standard is the charge of a commercially available version of the heparin or LMWH or is the charge of the heparin or LMWH prior to modification. In some embodiments, when the charge of the polysaccharide is neutralized, the net negative or net positive charge of the polysaccharide can be reduced by at least 10%, 20%, 30%, 40%, 50%, 60%, 70%, 80%, or 90%. In other embodiments, when the charge of the polysaccharide is neutralized, it can be neutralized such that there is a net negative and net positive charge of 0. The polysaccharide can be neutralized, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In other embodiments, when the charge of the polysaccharide is neutralized, it can be neutralized by contacting the polysaccharide with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In some embodiments, the polysaccharide has no N-sulfate groups or the polysaccharide that is less than 40%, 30%, 20%, 10% N-sulfated. In some embodiments, the polysaccharide is a heparin or LMWH that has been modified to decrease N-sulfation, e.g., the heparin or LMWH has been chemical or enzymatically treated to decrease N-sulfation of the heparin or LMWH, by at least 5%, 10%, 20%, 30%, 40%, 50%, 60%, 70%, 80%, 90%, 95% or more. In some embodiments, the heparin or LMWH is a heparin or LMWH is a heparin or LMWH described herein, e.g., a heparin or LMWH having a level of sulfation that has been modified by a method described herein.

In one aspect, the invention features a method of increasing the permeability of epithelial tissue. The method includes: contacting the epithelial tissue with a permeability increasing amount of a polysaccharide, e.g., a soluble polysaccharide, that has no N-sulfate groups or a polysaccharide that is less than 40%, 30%, 20%, 10% N-sulfated.

In one embodiment, the polysaccharide is a hexasaccharide or larger polysaccharide, e.g., an octasaccharide, decasaccharide or larger.

In one embodiment, the polysaccharide has been modified to decrease N-sulfation, e.g., the polysaccharide has been chemical or enzymatically treated to decrease N-sulfation of the polysaccharide, by at least 5%, 10%, 20%, 30%, 40%, 50%, 60%, 70%, 80%, 90%, 95% or more. For example, the polysaccharide has been, or can be, treated with pyridine and DMSO to decrease N-sulfation.

In some embodiments, the polysaccharide is a polysaccharide in which O-sulfation has not been modified.

In one embodiment, the polysaccharide is a glycoaminoglycan (GAG), e.g., a heparin-like glycoaminoglycan (HLGAG), or a heparin sulfate proteoglycan (HSPG).

In one embodiment, the polysaccharide is heparin or a fragment thereof, e.g., a LMWH, e.g., a LMWH is selected from the group consisting of enoxaparin, dalteparin, reviparin, tinzaparin, nadroparin, certoparin, ardeparin, M118 and parnaparin. In one embodiment, the polysaccharide is a LMWH having: one or more of the glucosamines that is N-acetylated; all of the glucosamines that are N-acetylated; none of the glucosamines that are N-acetylated; one or more of a 2-O sulfate, a 3-O sulfate and a 6-O sulfate.

In one embodiment, the LMWH has been modified to decrease N-sulfation, e.g., the polysaccharide has been chemical or enzymatically treated to decrease N-sulfation of the polysaccharide, by at least 5%, 10%, 20%, 30%, 40%, 50%, 60%, 70%, 80%, 90%, 95% or more. The LMWH can be modified to decrease sulfation, e.g., by a method described herein, e.g., the LMWH has been treated with pyridine and DMSO to decrease N-sulfation. In one embodiment, the LMWH has a molecular weight distribution and/or sugar composition of a selected LMWH reference, e.g., enoxaparin, dalteparin, reviparin, tinzaparin, nadroparin, certoparin, ardeparin, M118 and parnaparin. In one embodiment, the polysaccharide is in a preparation of polysaccharides, e.g., a preparation of polysaccharides comprising LMWH, e.g., a preparation comprising LMWH wherein all or a portion of the polysaccharides in the preparation consist of about two to twenty monosaccharides.

In one embodiment, the method further includes contacting the epithelial tissue with a therapeutic, prophylactic or diagnostic agent, e.g., a therapeutic, prophylactic or diagnostic agent described herein. In one embodiment, the agent can be a therapeutic or prophylactic polypeptide, nucleic acid, small molecule, lipid/glycolipids, etc. In one embodiment, the active agent is a therapeutic polypeptide selected from the group consisting of insulin, proinsulin, human growth hormone, interferon, α-1 proteinase inhibitor, alkaline phosphotase, angiogenin, cystic fibrosis transmembrane conductance regulator, extracellular superoxide dismutase, fibrinogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin. The therapeutic or prophylactic polypeptide can be an active derivative or fragment of such polypeptides. The active agent can also be, but is not limited to one or more of: parathyroid hormone and derivatives and fragments thereof, erythropoietin, epoetin beta, gene activated erythropoietin, second generation EPO, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, domase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/ somatropin, anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, sargramostim, lenograstim, molgramostim, mirimostim, nartograstim, oprelvekin, peptide tyrosin-tyrosin (PYY), apolipoprotein A-IV, leptin, melanocortin, amylin, orexin, adiponectin, and ghrelin. In one embodiment, the active agent is an active polypeptide, e.g., a therapeutic or prophylactic polypeptide, and the polypeptide has a molecular weight of about 150 kDa or less, about 100 kDa or less, about 50 kDa or less, or about 0.5-35 kDa or less. In one embodiment, the active agent is an active polypeptide, e.g., a therapeutic or prophylactic polypeptide, and the polypeptide has a molecular weight of about: 500 Da-5 kDa, 5 to 10 kDa, 10 to 30 kDa, 18 to 35 kDa, 30 to 50 kDa, 50 to 100 kDa, 100 to 150 kDa. In one embodiment, the active polypeptide is insulin or an active fragments or derivatives thereof. In another embodiment, the active polypeptide is human growth hormone or an active fragment or derivative thereof. In yet another embodiment, the active polypeptide is interferon.

In other embodiments, the agent is an inactive agent. Examples of inactive agents include biological probes or contrast agents for imaging.

In another embodiment, the agent can be a small molecule drug, e.g., a small molecule drug currently available for therapeutic, diagnostic, or prophylactic use, or a drug in development. In some embodiments, the agent is admixed with the polysaccharide. Admixtures can be prepared, e.g., by mixing, covalently-linked polysaccharides, ionically-linked polysaccharides, spraying drying and other techniques known in the art. In some embodiments, the agent is linked to one or more polysaccharides in the formulation. As an example, small molecule drugs, and protein-based drugs may be linked to polysaccharides for delivery via known chemistries such as EDC, CNBH4/DMSO/Acetic Acid, etc.

In one embodiment, the polysaccharide is a heparin or LMWH that has been modified to alter one or more of its charge, size, and/or therapeutic activity, e.g., by a method described herein. In one embodiment, the chemical signature of one or more polysaccharides in the preparation has been determined and, e.g., one or more polysaccharide is modified based upon its chemical signature. In some embodiments, the chemical signature is determined to evaluate whether the structure ΔUH_{NAc},6SGH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. is present or absent in the heparin or LMWH.

In one embodiment, the polysaccharide is a heparin or LMWH that has an anti-Xa activity and/or an anti-IIa activity that is reduced by at least 10%, 20%, 30%, 40%, 50%, 60%, 70% 80%, 90% or more as compared to a reference standard and, e.g., the reference standard is the level of anti-Xa activity and/or anti-IIa activity of a commercially available version of the heparin or LMWH or is the level of anti-Xa activity and/or anti-IIa activity of the heparin or LMWH prior to modification. In some embodiments, the heparin or LMWH is modified at one or more chemical signature of an oligosaccharide of heparin which comprises the structure ΔUHNAc,6SGHNS,3S,6S, ΔUHNS,6SGHNS,3S,6S, ΔUHNAc,6SGHNS,3S, and ΔUHNS,6SGHNS,3S, to reduce the anti-Xa activity and/or the anti-IIa activity of the heparin or LMWH. In some embodiments, the heparin or LMWH further has PF4 binding and/or FGF-2 binding that is reduced by at least 10%, 20%, 30%, 40%, 50%, 60%, 70% or more than a reference standard.

In one embodiment, the polysaccharide is a heparin or LMWH having a size that is reduced as compared to a reference standard, e.g., the reference standard is the level of average chain length of a commercially available version of the heparin or LMWH or is the average chain length of the heparin or LMWH prior to modification. In one embodiment, the mass of the polysaccharide is reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided polysaceharide. In other embodiments, the mass of the polysaccharide is reduced by at least 5, 10, 15, 20, 25, 30, 35, 40, 50, 60, 70, 80, 90, 100, 150, 200, 250, 300, 350, 500, 1000,1500, 2000, 2200, 2500, 3000 Da or more from the mass of the provided polysaccharide. In one embodiment, the mass of the provided polysaccharide can be reduced, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In one embodiment, the polysaccharide is a heparin or LMWH having a charge that has been modified as compared to a reference standard, e.g., the reference standard is the charge of a commercially available version of the heparin or LMWH or is the charge of the heparin or LMWH prior to modification. In some embodiments, when the charge of the polysaccharide is neutralized, the net negative or net positive charge of the polysaccharide can be reduced by at least 10%, 20%, 30%, 40%, 50%, 60%, 70%, 80%, or 90%. In other embodiments, when the charge of the polysaccharide is neutralized, it can be neutralized such that there is a net negative and net positive charge of 0. The polysaccharide can be neutralized, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In other embodiments, when the charge of the polysaccharide is neutralized, it can be neutralized by contacting the polysaccharide with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the polysaccharide is capable of increasing movement of the agent across an epithelium within 1, 5, 10, 15, 20, 25, 30 minutes after administration with the polysaccharide. In one embodiment, the polysaccharide is capable of increasing movement of the agent across an epithelium movement for a period of about 30 minutes to 5 hours, about 1 hour to 4 hours, about 2 hours to 3 hours. In one embodiment, the polysaccharide is contacted with the epithelial tissue prior to contacting the epithelial tissue with the agent, e.g., the agent is contacted with the epithelial tissue while the modulating effect of the polysaccharide is still detectable. In another embodiment, the agent is contacted with the epithelial tissue prior to contacting the epithelial tissue with the polysaccharide, e.g., the polysaccharide is contacted with the epithelial tissue while an effect of the agent is still detectable, e.g., the polysaccharide is contacted with the epithelial tissue such that movement of the agent across an epithelium is increased as compared to contacting the epithelial tissue with the agent in the absence of the polysaccharide. In another embodiment, the agent is contacted with the epithelial tissue simultaneously with the polysaccharide.

In one embodiment, the epithelial tissue includes pulmonary epithelial cells.

In another embodiment, the epithelial tissue includes ocular epithelial cells, dermal epithelial cells, nasal epithelial cells, vaginal epithelial cells, or epithelial cells of the mouth and/or throat.

In some embodiments, the polysaccharide and the agent are in a composition, e.g., a composition further comprising a pharmaceutically acceptable carrier and/or a delivery enhancer.

In one aspect, the invention features a method of delivering an effective amount of an agent to a subject, that includes administering to the subject a polysaccharide, e.g., a soluble polysaccharide, that has no N-sulfate groups or polysaccharide that is less than 40%, 30%, 20%, 10% N-sulfated, and an effective amount of the agent, to thereby deliver the agent to the subject.

In one embodiment, the polysaccharide is a hexasaccharide or larger polysaccharide, e.g., an octasaccharide, decasaccharide or larger.

In one embodiment, the polysaccharide has been modified to decrease N-sulfation, e.g., the polysaccharide has been chemical or enzymatically treated to decrease N-sulfation of the polysaccharide, by at least 5%, 10%, 20%, 30%, 40%, 50%, 60%, 70%, 80%, 90%, 95% or more. For example, the polysaccharide has been, or can be, treated with pyridine and DMSO to decrease N-sulfation.

In some embodiments, the polysaccharide is a polysaccharide in which O-sulfation has not been modified.

In one embodiment, the polysaccharide is a glycoaminoglycan (GAG), e.g., a heparin-like glycoaminoglycan (HLGAG), or a heparin sulfate proteoglycan (HSPG).

In one embodiment, the polysaccharide is heparin or a fragment thereof, e.g., a LMWH, e.g., a LMWH is selected from the group consisting of enoxaparin, dalteparin, reviparin, tinzaparin, nadroparin, certoparin, ardeparin, M118 and parnaparin. In one embodiment, the polysaccharide is a LMWH having: one or more of the glucosamines that is N-acetylated; all of the glucosamines that are N-acetylated; none of the glucosamines that are N-acetylated; one or more of a 2-O sulfate, a 3-O sulfate and a 6-O sulfate.

In one embodiment, the LMWH has been modified to decrease N-sulfation, e.g., the polysaccharide has been chemical or enzymatically treated to decrease N-sulfation of the polysaccharide, by at least 5%, 10%, 20%, 30%, 40%, 50%, 60%, 70%, 80%, 90%, 95% or more. The LMWH can be modified to decrease sulfation, e.g., by a method described herein, e.g., the LMWH has been treated with pyridine and DMSO to decrease N-sulfation. In one embodiment, the LMWH has a molecular weight distribution and/or sugar composition of a selected LMWH reference, e.g., enoxaparin, dalteparin, reviparin, tinzaparin, nadroparin, certoparin, ardeparin, M118 and parnaparin. In one embodiment, the polysaccharide is in a preparation of polysaccharides, e.g., a preparation of polysaccharides comprising LMWH, e.g., a preparation comprising LMWH wherein all or a portion of the polysaccharides in the preparation consist of about two to twenty monosaccharides.

In one embodiment, the method further includes contacting the epithelial tissue with a therapeutic, prophylactic or diagnostic agent, e.g., a therapeutic, prophylactic or diagnostic agent described herein. In one embodiment, the agent can be a therapeutic or prophylactic polypeptide, nucleic acid, small molecule, lipid/glycolipids, etc. In one embodiment, the active agent is a therapeutic polypeptide selected from the group consisting of insulin, proinsulin, human growth hormone, interferon, α-1 proteinase inhibitor, alkaline phosphotase, angiogenin, cystic fibrosis transmembrane conductance regulator, extracellular superoxide dismutase, fibrinogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and al-antitrypsin. The therapeutic or prophylactic polypeptide can be an active derivative or fragment of such polypeptides. The active agent can also be, but is not limited to one or more of: parathyroid hormone and derivatives and fragments thereof, erythropoietin, epoetin beta, gene activated erythropoietin, second generation EPO, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, dornase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/ somatropin, anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, sargramostim, lenograstim, molgramostim, mirimostim, nartograstim, oprelvekin, peptide tyrosin-tyrosin (PYY), apolipoprotein A-IV, leptin, melanocortin, amylin, orexin, adiponectin, and ghrelin. In one embodiment, the active agent is an active polypeptide, e.g., a therapeutic or prophylactic polypeptide, and the polypeptide has a molecular weight of about 150 kDa or less, about 100 kDa or less, about 50 kDa or less, or about 0.5-35 kDa or less. In one embodiment, the active agent is an active polypeptide, e.g., a therapeutic or prophylactic polypeptide, and the polypeptide has a molecular weight of about: 500 Da-5 kDa, 5 to 10 kDa, 10 to 30 kDa, 18 to 35 kDa, 30 to 50 kDa, 50 to 100 kDa, 100 to 150 kDa. In one embodiment, the active polypeptide is insulin or an active fragments or derivatives thereof. In another embodiment, the active polypeptide is human growth hormone or an active fragment or derivative thereof. In yet another embodiment, the active polypeptide is interferon.

In other embodiments, the agent is an inactive agent. Examples of inactive agents include biological probes or contrast agents for imaging.

In another embodiment, the agent can be a small molecule drug, e.g., a small molecule drug currently available for therapeutic, diagnostic, or prophylactic use, or a drug in development. In some embodiments, polysaccharide is admixed with the agent. Admixtures can be prepared, e.g., by mixing, covalently-linked polysaccharides, ionically-linked polysaccharides, spraying drying and other techniques known in the art.

In some embodiments, the agent is linked to one or more polysaccharides in the formulation. As an example, small molecule drugs, and protein-based drugs may be linked to polysaccharides for delivery via known chemistries such as EDC, CNBH4/DMSO/Acetic Acid, etc.

In one embodiment, the polysaccharide is a heparin or LMWH that has been modified to alter one or more of its charge, size, and/or therapeutic activity, e.g., by a method described herein. In one embodiment, the chemical signature of one or more polysaccharides in the preparation has been determined and, e.g., one or more polysaccharide is modified based upon its chemical signature. In some embodiments, the chemical signature is determined to evaluate whether the structure ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. is present or absent in the heparin or LMWH.

In one embodiment, the polysaccharide is a heparin or LMWH that has an anti-Xa activity and/or an anti-IIa activity that is reduced by at least 10%, 20%, 30%, 40%, 50%, 60%, 70% 80%, 90% or more as compared to a reference standard and, e.g., the reference standard is the level of anti-Xa activity and/or anti-IIa activity of a commercially available version of the heparin or LMWH or is the level of anti-Xa activity and/or anti-IIa activity of the heparin or LMWH prior to modification. In some embodiments, the heparin or LMWH is modified at one or more chemical signature of an oligosaccharide of heparin which comprises the structure ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. to reduce the anti-Xa activity and/or the anti-IIa activity of the heparin or LMWH. In some embodiments, the heparin or LMWH further has PF4 binding and/or FGF-2 binding that is reduced by at least 10%, 20%, 30%, 40%, 50%, 60%, 70% or more than a reference standard.

In one embodiment, the polysaccharide is a heparin or LMWH having a size that is reduced as compared to a reference standard, e.g., the reference standard is the level of average chain length of a commercially available version of the heparin or LMWH or is the average chain length of the heparin or LMWH prior to modification. In one embodiment, the mass of the polysaccharide is reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided polysaccharide. In other embodiments, the mass of the polysaccharide is reduced by at least 5, 10, 15, 20, 25, 30, 35, 40, 50, 60, 70, 80, 90, 100, 150, 200, 250, 300, 350, 500, 1000,1500, 2000, 2200, 2500, 3000 Da or more from the mass of the provided polysaccharide. In one embodiment, the mass of the provided polysaccharide can be reduced, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In one embodiment, the polysaccharide is a heparin or LMWH having a charge that has been modified as compared to a reference standard, e.g., the reference standard is the charge of a commercially available version of the heparin or LMWH or is the charge of the heparin or LMWH prior to modification. In some embodiments, when the charge of the polysaccharide is neutralized, the net negative or net positive charge of the polysaccharide can be reduced by at least 10%, 20%, 30%, 40%, 50%, 60%, 70%, 80%, or 90%. In other embodiments, when the charge of the polysaccharide is neutralized, it can be neutralized such that there is a net negative and net positive charge of 0. The polysaccharide can be neutralized, e.g., by digesting the polypeptide with at least one agent, e.g., an agent selected based upon the chemical signature of the polysaccharide. For example, the agent can be an enzyme (e.g., an enzyme which is capable of cleaving the polysaccharide at known locations in the polysaccharide based upon its chemical signature) or a chemical (e.g., a chemical capable of cleaving the polysaccharide at known locations in the polysaccharide based upon its chemical signature) or combinations thereof. Examples of enzymes which can be used include heparin degradation enzymes, e.g., heparin lysase such as heparinase I, heparinase II, heparinase III, heparinase IV, heparanase, and functionally active fragments and variants thereof. Examples of chemicals which can be used include oxidative depolymerization with H2O2 or Cu+ and H2O2, deaminative cleavage with isoamyl nitrite, or nitrous acid, eliminative cleavage with benzyl ester of heparin by alkaline treatment or by heparinase.

In other embodiments, when the charge of the polysaccharide is neutralized, it can be neutralized by contacting the polysaccharide with a charge neutralizing agent, e.g., a counter ion such as mono- or divalent ion, (e.g., barium, calcium, sodium, potassium, lithium, ammonium, magnesium, zinc), a transition metal (e.g., iron, nickel and copper), and/or other neutralizing compounds (e.g., a small organic compound, spermine, spermidine, low molecular weight protamine, basic peptides).

In one embodiment, the polysaccharide is capable of administering the agent within 1, 5, 10, 15, 20, 25, 30 minutes after administration with the polysaccharide, e.g., as compared to the administration in the absence of the polysaccharide. In one embodiment, the polysaccharide is capable of increasing the delivery of the agent for a period of about 30 minutes to 5 hours, about 1 hour to 4 hours, about 2 hours to 3 hours, e.g., as compared to the delivery of the agent in the absence of the polysaccharide. In one embodiment, the polysaccharide is administered prior to administration of the agent, e.g., the agent is administered while the modulating effect of the polysaccharide is still detectable. In another embodiment, the agent is administration prior to administration of the polysaccharide, e.g., the polysaccharide is administered while an effect of the agent is still detectable, e.g., the polysaccharide is contacted is administered such that delivery of the agent is increased or decreased as compared to administration of the agent in the absence of the polysaccharide. In another embodiment, the agent is administered simultaneously with the polysaccharide.

In some embodiments, the polysaccharide and the agent are in a composition, e.g., a composition further comprising a pharmaceutically acceptable carrier and/or a delivery enhancer.

In some embodiments, the agent and polysaccharide are administered: by pulmonary delivery; by oral delivery; by topical delivery, e.g., to the skin, the rectum, the vagina, the eye. In some embodiments, the agent is delivered to the lung, eye, nose, throat, gastrointestinal tract, or skin.

In another aspect, the invention features a formulation for delivery of a therapeutic, prophylactic or diagnostic agent that includes an effective amount of the agent and a polysaccharide, e.g., a soluble polysaccharide, that has no N-sulfate groups or a polysaccharide that is less than 40%, 30%, 20%, 10% N-sulfated. The agent and/or polysaccharide can be, e.g., an agent or polysaccharide described herein.

In one embodiment, the polysaccharide is a hexasaccharide or larger polysaccharide, e.g., an octasaccharide, decasaccharide or larger.

In one embodiment, the polysaccharide has been modified to decrease N- sulfation, e.g., the polysaccharide has been chemical or enzymatically treated to decrease N-sulfation of the polysaccharide, by at least 5%, 10%, 20%, 30%, 40%, 50%, 60%, 70%, 80%, 90%, 95% or more. For example, the polysaccharide has been treated with pyridine and DMSO to decrease N-sulfation. In one embodiment, the LMWH has a molecular weight distribution and/or sugar composition of a selected LMWH reference, e.g., enoxaparin, dalteparin, reviparin, tinzaparin, nadroparin, certoparin, ardeparin, M118 and parnaparin. In one embodiment, the polysaccharide is in a preparation of polysaccharides, e.g., a preparation of polysaccharides comprising LMWH, e.g., a preparation comprising LMWH wherein all or a portion of the polysaccharides in the preparation consist of about two to twenty monosaccharides.

In some embodiments, the polysaccharide is a polysaccharide in which O- sulfation has not been modified.

In one embodiment, the polysaccharide is a glycoaminoglycan (GAG), e.g., a heparin-like glycoaminoglycan (HLGAG), or a heparin sulfate proteoglycan (HSPG).

In one embodiment, the polysaccharide is heparin or a fragment thereof, e.g., a LMWH, e.g., a LMWH is selected from the group consisting of enoxaparin, dalteparin, reviparin, tinzaparin, nadroparin, certoparin, ardeparin, M118 and parnaparin. In one embodiment, the polysaccharide is a LMWH having: one or more of the glucosamines that is N-acetylated; all of the glucosamines that are N-acetylated; none of the glucosamines that are N-acetylated; one or more of a 2-O sulfate, a 3-O sulfate and a 6-O sulfate.

In one embodiment, the agent can be a therapeutic or prophylactic polypeptide, nucleic acid, small molecule, lipid/glycolipids, etc. In one embodiment, the active agent is a therapeutic polypeptide selected from the group consisting of insulin, proinsulin, human growth hormone, interferon, α-1 proteinase inhibitor, alkaline phosphotase, angiogenin, cystic fibrosis transmembrane conductance regulator, extracellular superoxide dismutase, fibrinogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin. The therapeutic or prophylactic polypeptide can be an active derivative or fragment of such polypeptides. The active agent can also be, but is not limited to one or more of: parathyroid hormone and derivatives and fragments thereof, erythropoietin, epoetin beta, gene activated erythropoietin, second generation EPO, novel erythropoiesis stimulating protein, insulin lispro, insulin (bovine), insulin, insulin aspart, insulin analogue, Calcitonin, Theraccine, becaplermin (recombinant human platelet derived growth factor-BB), trafernin, human growth hormone-releasing factor, BMP-7, PEG aspariginase, domase alpha, alglucerase, agalsidase-beta, dornase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/somatropin, anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, sargramostim, lenograstim, molgramostim, mirimostim, nartograstim, oprelvekin, peptide tyrosin-tyrosin (PYY), apolipoprotein A-IV, leptin, melanocortin, amylin, orexin, adiponectin, and ghrelin. In one embodiment, the active agent is an active polypeptide, e.g., a therapeutic or prophylactic polypeptide, and the polypeptide has a molecular weight of about 150 kDa or less, about 100 kDa or less, about 50 kDa or less, or about 0.5-35 kDa or less. In one embodiment, the active agent is an active polypeptide, e.g., a therapeutic or prophylactic polypeptide, and the polypeptide has a molecular weight of about: 500 Da-5 kDa, 5 to 10 kDa, 10 to 30 kDa, 18 to 35 kDa, 30 to 50 kDa, 50 to 100 kDa, 100 to 150 kDa. In one embodiment, the active polypeptide is insulin or an active fragments or derivatives thereof. In another embodiment, the active polypeptide is human growth hormone or an active fragment or derivative thereof. In yet another embodiment, the active polypeptide is interferon.

In other embodiments, the agent is an inactive agent. Examples of inactive agents include biological probes or contrast agents for imaging.

In another embodiment, the agent can be a small molecule drug, e.g., a small molecule drug currently available for therapeutic, diagnostic, or prophylactic use, or a drug in development. In some embodiments, polysaccharide is admixed with the agent. Admixtures can be prepared, e.g., by mixing, covalently-linked polysaccharides, ionically-linked polysaccharides, spraying drying and other techniques known in the art.

In some embodiments, the agent is linked to one or more polysaccharides in the formulation. As an example, small molecule drugs, and protein-based drugs may be linked to polysaccharides for delivery via known chemistries such as EDC, CNBH4/DMSO/Acetic Acid, etc.

In one embodiment, the polysaccharide is a heparin or LMWH that has been modified to alter one or more of its charge, size, and/or therapeutic activity, e.g., by a method described herein. In one embodiment, the chemical signature of one or more polysaccharides in the preparation has been determined and, e.g., one or more polysaccharide is modified based upon its chemical signature. In some embodiments, the chemical signature is determined to evaluate whether the structure ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S} is present or absent in the heparin or LMWH.

In one embodiment, the polysaccharide is a heparin or LMWH that has an anti-Xa activity and/or an anti-IIa activity that is reduced by at least 10%, 20%, 30%, 40%, 50%, 60%, 70% 80%, 90% or more as compared to a reference standard and, e.g., the reference standard is the level of anti-Xa activity and/or anti-IIa activity of a commercially available version of the heparin or LMWH or is the level of anti-Xa activity and/or anti-IIa activity of the heparin or LMWH prior to modification. In some embodiments, the heparin or LMWH has been modified at one or more chemical signature of an oligosaccharide of heparin which comprises the structure ΔUHNAc,6SGHNS,3S,6S, ΔUHNS,6SGHNS,3S,6S, ΔUHNAc,6SGHNS,3S, and ΔUHNS,6SGHNS,3S, to reduce the anti-Xa activity and/or the anti-IIa activity of the heparin or LMWH. In some embodiments, the heparin or LMWH further has PF4 binding and/or FGF-2 binding that has been reduced by at least 10%, 20%, 30%, 40%, 50%, 60%, 70% or more than a reference standard. In one embodiment, the polysaccharide is a LMWH and the LMWH has on a weight per weight basis less than X% of the anti-Xa activity and/or anti-IIa activity as compared to a reference, e.g., enoxaparin, dalteparin, reviparin, tinzaparin, nadroparin, certoparin, ardeparin, M118 and parnaparin.

In one embodiment, the polysaccharide is a heparin or LMWH having a size that is reduced as compared to a reference standard, e.g., the reference standard is the level of average chain length of a commercially available version of the heparin or LMWH or is the average chain length of the heparin or LMWH prior to modification. In one embodiment, the mass of the polysaccharide is reduced by at least 5%, 10%, 15%, 20%, 25%, 30%, 35%, 40%, 45%, 50%, 55%, 60%, 65%, 70%, 75%, 80%, 85%, or 90% (and integers there between) from the mass of the provided polysaccharide. In other embodiments, the mass of the polysaccharide is reduced by at least 5, 10, 15, 20, 25, 30, 35, 40, 50, 60, 70, 80, 90, 100,150, 200, 250, 300, 350, 500, 1000, 1500, 2000, 2200, 2500, 3000 Da or more from the mass of the provided polysaccharide.

In one embodiment, the polysaccharide is a heparin or LMWH having a charge that has been modified as compared to a reference standard, e.g., the reference standard is the charge of a commercially available version of the heparin or LMWH or is the charge of the heparin or LMWH prior to modification. In some embodiments, when the charge of the polysaccharide is neutralized, the net negative or net positive charge of the polysaccharide can be reduced by at least 10%, 20%, 30%, 40%, 50%, 60%, 70%, 80%, or 90%. In other embodiments, when the charge of the polysaccharide is neutralized, it can be neutralized such that there is a net negative and net positive charge of 0.

In one embodiment, the formulation further includes one or more of: a pharmaceutically acceptable carrier and a delivery enhancer, e.g., one or more of a surfactant, an absorption enhancer, protease inhibitor, etc.

In one embodiment, the formulation is for pulmonary delivery and the formulation is provided in a device for pulmonary delivery, e.g., a pressurized container or dispenser e.g., a pressurized contained or dispenser which contains a suitable propellant and/or nebulizer, or is user activated. In one embodiment, the formulation is provided in a delivery device for pulmonary delivery that delivers a metered dose of the formulation to a subject.

In one embodiment, the formulation is a dry formulation, e.g., a dry formulation that includes LMWH particles having a mean geometric diameter of 1 to 500 microns, e.g., at least 2 to 100 microns. In some embodiments, the dry formulation includes polysaccharide particles having a mean geometric diameter of at least 2, 3, 4, 5, 10, 15, 20, 25, 30, 35, 40, 45, 50 or 100 microns. In one embodiment, the formulation is a liquid formulation, an aerosol, a mist, or a suspension.

In one embodiment, the formulation is for topical delivery and the formulation is provided in a device for topical delivery, e.g., a transdermal patch, a cream, an ointment, a suppository.

In another aspect, the invention features a formulation for affecting movement of the agent across an epithelium movement of an agent comprising a polysaccharide and an agent, wherein the polysaccharide is capable of modulating intercellular junctions between mammalian epithelial cells, e.g., the polysaccharide and/or agent is a polysaccharide and/or agent described herein.

In another aspect, the invention features a method of evaluating a polysaccharide to determine if the polysaccharide will modulate permeability of epithelial tissue. The method includes: providing an intercellular junction protein, e.g., a protein selected from a claudin, a junction associate molecule (JAM), an occludin, and a zona occluden, e.g., a zona occludin, ZO-1, ZO-2 or ZO-3; contacting the protein with a polysaccharide; and determining if the polysaccharide interacts with, e.g., binds to, the protein, wherein interaction of the polysaccharide with the protein is indicative of a polysaccharide that modulates the permeability of epithelial tissue.

In another aspect, the invention features a method of evaluating a polysaccharide to determine if the polysaccharide will modulate permeability of epithelial tissue. The method includes: administering a detectable agent, e.g., an antibody or ligand, that interacts with, e.g., binds to, an intercellular junction protein, e.g., a protein selected from a claudin, a junction associate molecule (JAM), an occludin, and a zona occluden, e.g., a zona occludin, ZO-1, ZO-2 or ZO-3 to a subject; administering a polysaccharide, e.g., a detectably labeled polysaccharide, to the subject;

detecting the detectable agent that interacts with, e.g., binds to, the intercellular junction protein and the polysaccharide to determine whether the intercellular junction protein and the polysaccharide are associated with each other, wherein association of the polysaccharide with the protein is indicative of a polysaccharide that modulates the permeability of epithelial tissue.

An agent that binds to the intercellular junction protein and/or the polysaccharide can be, e.g., directly or indirectly labeled.

In another aspect, the invention features methods of preparing a polysaccharide (e.g., a polysaccharide described herein), and/or a formulation of a polysaccharide and an agent (e.g., a formulation described herein), e.g., by methods described herein.

In some embodiments, the heparin is one in which one or more chemical signatures of a oligosaccharide of the heparin that include the structure: ΔUH_{NAc,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}, is modified to reduce the anti-Xa activity of the heparin, e.g., the heparin include one or more of ΔUH_{NAc,6S}GH_{NS,3S,6S} or ΔUH_{NAc,6S}GH_{NS,3S}. In some embodiments, the heparin is M118, which has a molecular weight of 5,000 Da, a polydispersity of 1.0, and a higher weight percent of peak 8 than other LMWHs. M118 is a LMWH having XA and IIA activity on the same molecule and it is fully neutralizable by protamine.

In some embodiments, one or more monosaccharide or disaccharide is added or removed, and/or one or more acetyl group and/or sulfo group is substituted, removed or added, to modify the activity of the heparin.

### BRIEF DESCRIPTION OF THE FIGURES

FIG. 1A-C: ZO-1 staining in Control (non-treated naïve) rat upper lung epithelium. A. Minimal upper lung tissue autofluorescence in FITC channel; B. ZO-1 localization (as detected by Cy3 secondary antibody) in upper lung epithelium; C. Overlay.

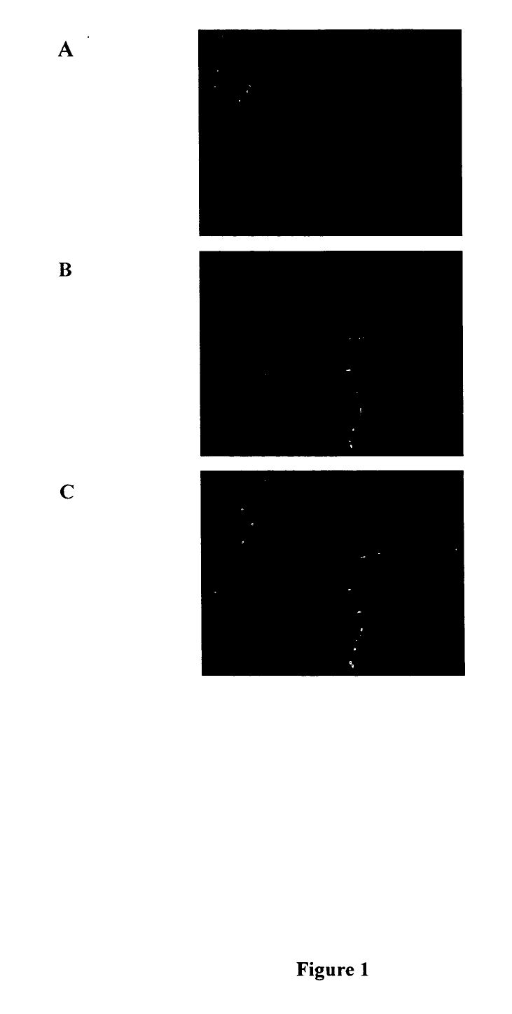

FIG. 2A-C: ZO-1 staining in control (non-treated, naïve) rat deep lung epithelium. A. Minimal deep lung tissue autofluorescence in FITC channel; B. ZO-1 localization (as detected by Cy3 secondary antibody) in deep lung epithelium; C. Overlay.

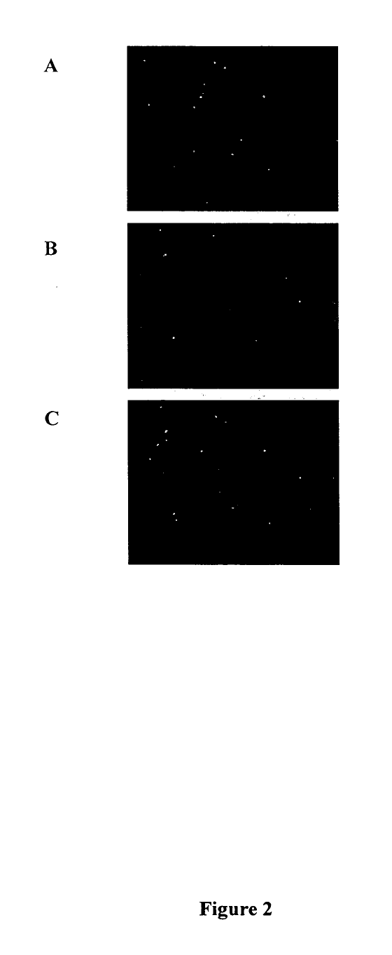

FIG. 3A-C: ZO-1 staining in rat upper lung epithelium after fluorescein-ardeparin treatment, 30 min. A. Localization of fluorescein-Ard in airway overlying lung epithelium; B. ZO-1 localization (as detected by Cy3 secondary antibody) in lung epithelium; C. Overlay.

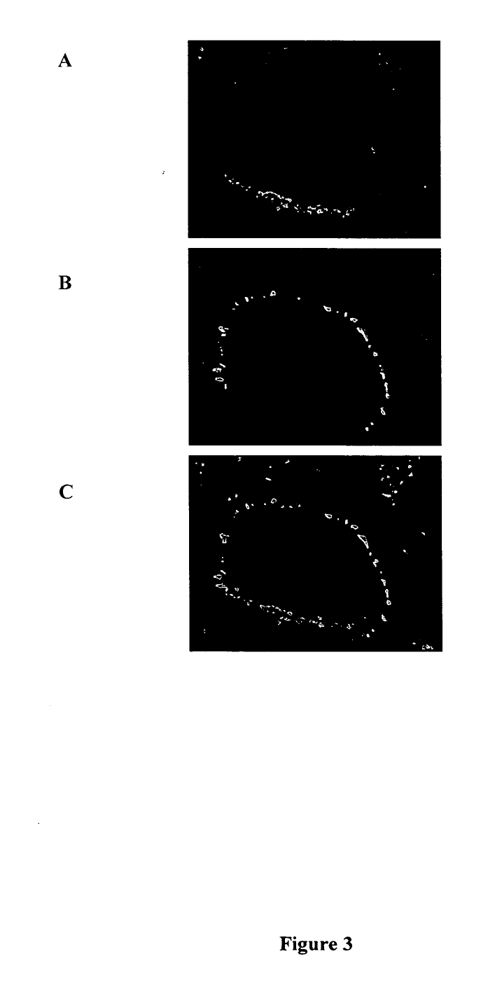

FIG. 4A-C: ZO-1 staining of non-treated area in rat upper lung epithelium after fluorescein-ardeparin treatment, 30 minutes. A. Minimal lung tissue autofluorescence in FITC channel B. ZO-1 localization (as detected by Cy3 secondary antibody) in lung epithelium C. Overlay.

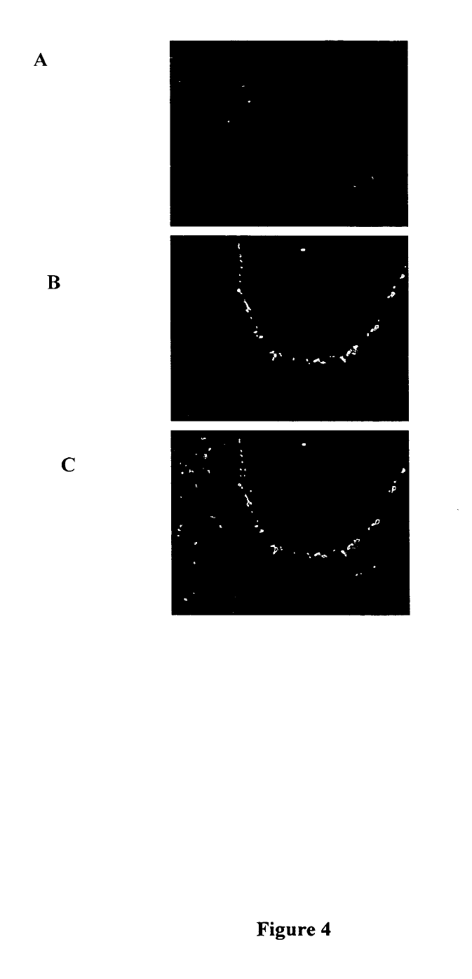

FIG. 5A-C: ZO-1 staining in rat upper lung epithelium after FITC-insulin/lactose treatment, 60 minutes. A. Localization of FITC-insulin/lactose in airway overlying lung epithelium; B. ZO-1 localization (as detected by Cy3 secondary antibody) in lung epithelium; C. Overlay.

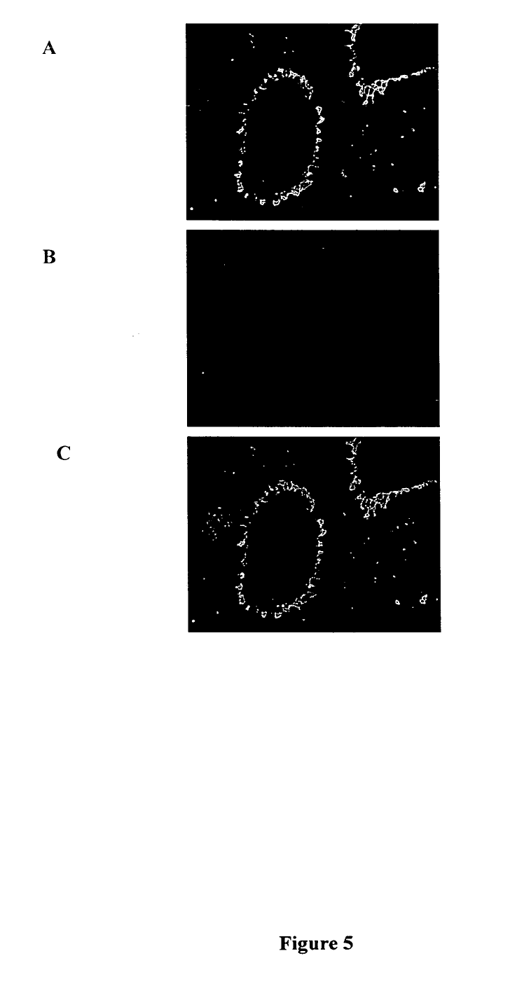

FIG. 6A-C: ZO-1 staining in rat upper lung epithelium after FITC-insulin/ardeparin treatment, 10 minutes. A. Localization of FITC-insulin/ardeparin in airway overlying lung epithelium; B. ZO-1 localization (as detected by Cy3 secondary antibody) in lung epithelium; C. Overlay.

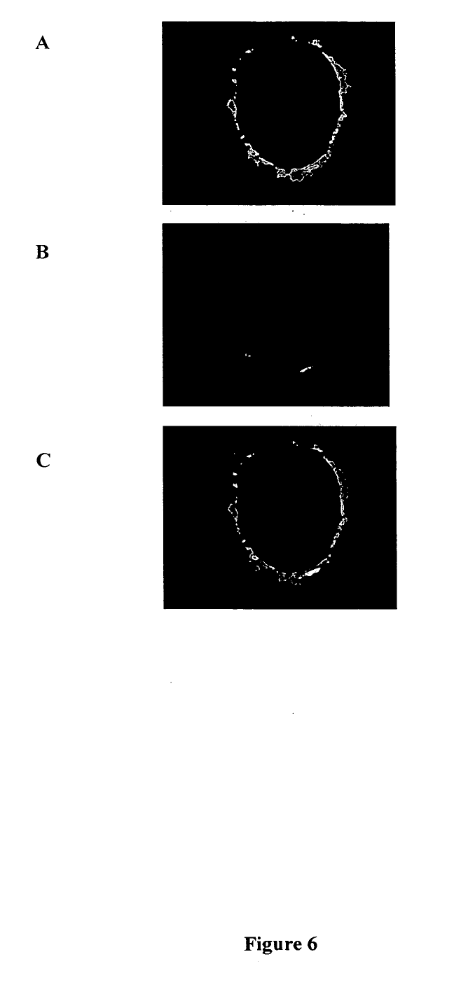

FIG. 7A-C: Summary of ZO-1 staining in rat upper lung epithelium after various treatments. A. Fluorescein-Ardeparin, 30 minutes; B. FITC-Insulin/ardeparin, 10 minutes; C. FITC-Insulin/lactose, 60 minutes.

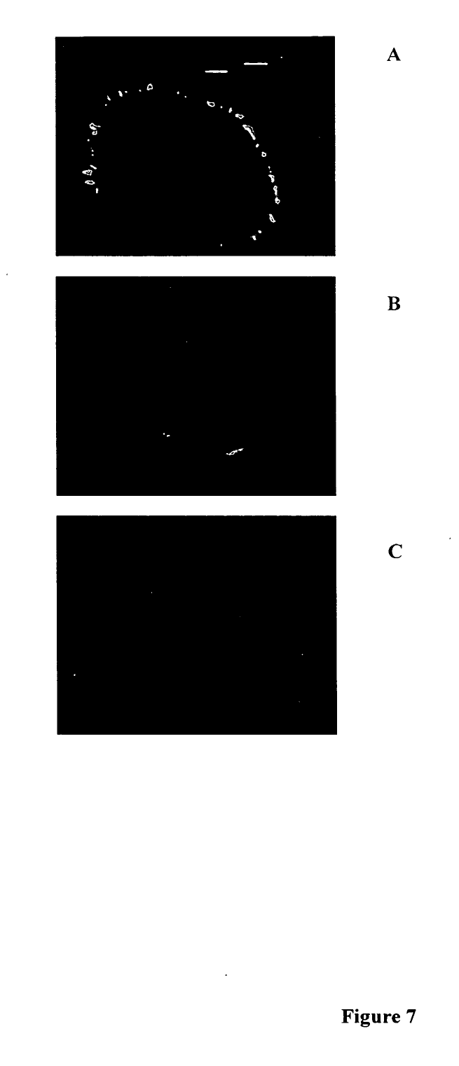

### DETAILED DESCRIPTION OF THE INVENTION

It was discovered that polysaccharides such as heparin and low molecular weight heparin (LMWH) deliver biological agents, regardless of the size of the biological agent, through epithelial barriers encountered by various routes of delivery. It was found that polysaccharides can modulate intercellular junctions between epithelial cells, e.g., by modulating expression and/or activity of one or more junction proteins, e.g., zonula occludins-1 (ZO-1). For example, it was found that polysaccharides having one or more of the following characteristics: little or low levels of N-sulfation, decreased activity, and a reduced mass; provided enhanced delivery of biological agents across epithelial tissue as compared to the same polysaccharide without one or more of these characteristics.

The methods described herein can be used to deliver biological agents at high levels of bioavailability by administrative routes that in the past had met with limited success. As shown in the Figures, compositions that include a LMWH and a biologically agent, namely insulin, have been generated which have enhanced pulmonary delivery profiles. In addition, such compositions can also be used for other routes of administration such as ocular delivery, topical delivery, mucosal and non-mucosal delivery.

A “polysaccharide” as used herein is a polymer composed of monosaccharides linked to one another. In many polysaccharides, the basic building block of the polysaccharide is actually a disaccharide unit, which can be repeating or non-repeating. Thus, a unit when used with respect to a polysaccharide refers to a basic building block of a polysaccharide and can include a monomeric building block (monosaccharide) or a dimeric building block (disaccharide). Polysaccharides include but are not limited to heparin-like glycosaminoglycans and derivatives and analogs thereof, chondroitin sulfate and derivatives and analogs thereof, hyaluronic acid and derivatives or analogs thereof, dermatan sulfate and derivatives or analogs thereof, keratan sulfate and derivatives or analogs thereof, chitin derivatives and analogs thereof, e.g., 6-0-sulfated carboxymethyl chitin, chitosan and derivatives or analogs thereof, immunogenic polysaccharides isolated from phellinus linteus, PI-88 (a mixture of highly sulfated oligosaccharide derived from the sulfation of phosphomannum which is purified from the high molecular weight core produced by fermentation of the yeast pichia holstii) and its derivatives and analogs, polysaccharide antigens for vaccines, and calcium spirulan (Ca-SP, isolated from blue-green algae, spirulina platensis) and derivatives and analogs thereof.

One preferred type of polysaccharide is an HLGAG. Thus, in some embodiments, the polysaccharide is a heparin-like glycosaminoglycan (HLGAG) and, e.g., HLGAG that has no or low levels of N-sulfation (e.g., as compared to a reference standard) and/or that has a reduced activity and/or molecular weight (e.g., a compared to a reference standard. The methods taught herein are sometimes described with reference to HLGAGs but the properties taught herein can be extended to other polysaccharides, and unless a claim specifies otherwise the claims encompass any polysaccharide. As used herein the terms “HLGAG” and “glycosaminoglycans” are used interchangeably to refer to a family of molecules having heparin like structures and properties. These molecules include but are not limited to low molecular weight.heparin (LMWH), heparin, biotechnologically prepared heparin, chemically modified heparin, synthetic heparins, heparin mimetics and heparan sulfate. The term “biotechnological heparin” encompasses heparin that is prepared from natural sources of polysaccharides which have been chemically modified and is described in Razi et al., Bioche. J. 1995 July 15;309 (Pt 2): 465-72. Chemically modified heparin is described in Yates et al., Carbohydrate Res (1996) November 20;294:15-27, and is known to those of skill in the art. Synthetic heparin is well known to those of skill in the art. Heparan Sulfate refers to a glycosaminoglycan containing a disaccharide repeat unit similar to heparin, but which has more N-acetyl groups and fewer N- and O-sulfate groups. Heparin mimetics are monosaccharides (e.g., sucralfate), oligosaccharides, or polysaccharides having at least one biological activity of heparin (i.e., anticoagulation, inhibition of cancer, treatment of lung disorders, etc.) or structurally similar properties of heparin. Heparin mimetics may be naturally occurring, synthetic or chemically modified. (Barchi, J.J., Curr. Pharm. Des., 2000, March, 6(4):485-501). The term “HLGAG” also encompasses functional variants of the above-described HLGAG molecules. These functional variants have a similar structure but include slight modifications to the structure.

“LMWH” as used herein refers to a preparation of sulfated glycosaminoglycans (GAGs) having an average molecular weight of less than 8000 Da, with about at least 60% of the oligosaccharide chains of a LMWH preparation having a molecular weight of less than 8000 Da. Several LMWH preparations are commercially available, but, LMWHs can also be prepared from heparin, using e.g., HLGAG degrading enzymes. HLGAG degrading enzymes include but are not limited to heparinase-I, heparinase-II, heparinase-III, heparinase IV, heparanase, D-glucuronidase and L-iduronidase. The three heparinases from Flavobacterium heparinum are enzymatic tools that have been used for the generation of LMWH (5,000-8,000 Da) and ultra-low molecular weight heparin (˜3,000 Da). Commercially available LMWH include, but are not limited to, enoxaparin (brand name Lovenox, Aventis Pharmaceuticals; other enoxaparins include those made by Opocrin, Gland, Enorin), dalteparin (Fragmin, Pharmacia and Upjohn), certoparin (Sandobarin, Novartis), ardeparin (Normiflo, Wyeth Lederle), nadroparin (Fraxiparine, Sanofi-Winthrop), parnaparin (Fluxum, Wassermann), reviparin (Clivarin, Knoll AG), and tinzaparin (Innohep, Leo Laboratories, Logiparin, Novo Nordisk). Some preferred forms of LMWH include enoxaparin (Lovenox) and dalteparin (Fragmin). A “synthetic heparin” or “synthetic HLGAG” as used herein refers to HLGAGs that are synthesized compounds and are not derived by fragmentation of heparin. Methods of preparing synthetic heparins are provided, for example, in Petitou et al. (1999) Nature 398:417, the contents of which is incorporated herein by reference. The term synthetic heparins also include derivatives thereof.

Modulation of the permeability of intercellular tight junctions, as used herein, refers to alterations of the native state of tight junction components associated with transport of agents across tight junctions. This can include disruption and reassembly of intercellular junction components, depletion of intracellular junction components, and synthesis of intercellular junction components. When the permeability of intercellular tight junctions is decreased, the ability of agents to cross the junction is decreased. When the permeability of intercellular tight junctions is increased, the ability of agents to cross the junction is increased.

Methods of Desulfating a Polysaccharide

The formulations and methods described herein use a polysaccharide to modulate the permeability of epithelial tissue by, e.g., modulating the permeability of intercellular junctions such as tight junctions. In some embodiments, the formulations and methods increase the permeability of epithelial tissue using a polysaccharide that can alter the native state of the tight junction. Polysaccharides having this property include, e.g., polysaccharides that have no N-sulfate groups or that are less than 40%, 30%, 20%, 10%, 5% N-sulfated. The level of sulfation of a polysaccharide can be decreased by chemically or enzymatically treating the polysaccharide. Various methods of desulfation such as N-desulfation are known in the art. For example, treatment of a polysaccharide such as heparin and LMWHs with pyridine and DMSO can be used to remove sulfates, e.g., N-sulfates. Inoue et al. (1976) Carbohydrate Research 46(1):87-95 and Nagasawa et al. (1980) Methods of Carbohydrate Chemistry VIII:291-294. The conditions can be adjusted such that the polysaccharide is completely desulfated, or partially desulfated, at one or more N- or O-sulfate (e.g., a 2-O sulfate, a 3-O sulfate and/or a 6-O sulfate) of the polysaccharide.

In addition, a polysaccharide for use in the formulations and methods of the invention can be, wholly or partially N-desulfated, without modifying O-sulfation of the polysaccharide. Methods are known for, at least partially, N-desulfating a polysaccharide such as heparin or LMWHs without significantly altering O-sulfation of the polysaccharide. For example, dilute acid can be used for removing or decreasing the level of N-sulfation of a polysaccharide without significantly altering O-sulfation.

Various methods have also been described for decreasing the level of N-sulfation or removing N-sulfation from a polysaccharide such as heparin and LMWHs and then acetylating at least a portion of the desulfated N-positions of the polysaccharide. Such methods can provide, e.g., a heparin or LMWH in which one, some or all of the glucosamines in the heparin or LMWH are N-acetylated. For example, Katachkine et al. (1981) J. Clin. Invest. 67:223-2238 describe methods of N-acetylating an N-desulfated heparin by reacting N-desulfated or partially N-desulfated heparin with excess ¹⁴C acetic anhydrate at pH 7.5. See also, e.g., Mulloy et al (1994) Carbohydrate Research 255:1-26.

Moreover, N-sulfation and/or O-sulfation of a polysaccharide can be altered using enzymatic means 2-O sulfatase, 3-O sulfatase, 6-O sulfatase, N-sulfatase, glucosamine 6-O sulfatase, N sulfamidase and their derivatives.

Methods that can be used to determine the level of sulfation and/or the types of sulfation of a polysaccharide and methods that can be used to test the activity of the modified polysaccharide are described below.

Methods of Reducing a Biological Activity of a Polysaccharide

The polysaccharides for use in the formulations and methods described herein can be modified to reduce one or more therapeutic activity of the polysaccharide. For example, a heparin can be modified such that one or more activity against Factor Xa and Factor IIa in blood coagulation is reduced. Such polysaccharides can be used, e.g., to increase the permeability of epithelial tissue thereby enhancing permeability across epithelial barriers.

Polysaccharides can be modified to reduce the therapeutic actions of the polysaccharide, e.g., by reducing the net charge, mass and/or size of the polysaccharide. These modifications can be made either enzymatically or chemically, e.g., as described herein. The resulting activity can then be determined using standard chromogenic assays.

For Xa of a heparin, the activity can be based on a sequence of a oligosaccharide comprising peak 8 or a tetrasaccharide within that structure. There are several ways to reduce anti-Xa activity of a heparin. For example, one or more of the following can be done to a heparin that includes the structures ΔUH_{NAC,6S}GH_{NS,3S,6S}, ΔUH_{NS,6S}GH_{NS,3S,6S}, ΔUH_{NAc,6S}GH_{NS,3S}, or ΔUH_{NS,6S}GH_{NS,3S}: lower the sulfation, modify the functional groups with non-sulfates, and reduce the size of the chain to below the oligosaccharide. Specifically, removal of 2-O, 3-O, 6-O, and/or N-sulfates, in various combinations, can be used to completely, or in partially, reduce the anti-Xa activity of a heparin.

For IIa of a heparin, that activity can be based on an octadecasaccharide (18-) that also contains the peak 8. Thus, the same approaches can be used as for reducing anti-IIa activity as is described for reducing anti-Xa activity. The approaches include decreasing the molecular weight/size of the chain.

Methods that can be used to modulate the activity of a polysaccharide and methods that can be used to test the activity of the modified polysaccharide are described below.

Methods of Determining the Chemical Signature of a Polysaccharide and Methods of Modulating Activity, Charge Level of Sulfation and/or Size of a Polysaccharide

In addition to modifying the polysaccharide to reduce a therapeutic activity of the polysaccharide, the polysaccharide can also be modified to alter the net charge, mass and/or size of the polysaccharide. The delivery profiles, described herein, can be further enhanced, e.g., by neutralizing a polysaccharide, adding charged elements to a polysaccharide, and/or reducing the mass of the polysaccharide, and/or the mass of the structure. For example, using the chemical signature of the polysaccharide, charges can be modulated, e.g., neutralized or enhanced, and/or the size of the polysaccharide reduced. In one embodiment, the chemical signature can be used to determine the level of sulfation, e.g., N-sulfation, of the polysaccharide.

A “neutralized formulation” as used herein is a formulation in which the net negative or positive charge has been reduced or masked by at least 10%. In other embodiments, the neutralized formulation is a formulation in which the net negative or positive charge has been reduced by at least 20%, 30%, 40%, 50%, 60%, 70%, 80, 90% or 100% or any integer there between. A “completely neutral” formulation is one in which there is a net negative and positive charge of zero.

Specific chemical properties of a polysaccharide may be identified and manipulated in order to reduce a specific therapeutic activity of the polysaccharide and/or enhance delivery of one or more active agent(s) by various routes of administration. In other embodiments, the specific chemical properties of a polysaccharide can be identified and manipulated in order to decrease permeability across epithelial tissue. The chemical properties of the polysaccharide may be altered by various techniques in order to reduce the biological activity of the polysaccharide and/or enhance delivery of an active agent (e.g., a therapeutic, prophylactic or diagnostic agent) associated with polysaccharides or to decrease delivery of an agent across an epithelia barrier. Methodologies have been developed to determine chemical signatures of polysaccharides. A chemical signature, as used herein, refers to information regarding, e.g., the identity and number the mono- and disaccharide building blocks of a polysaccharide, information regarding the physiochemical properties such as the overall (also referred to as the “net charge”), charge density, molecular size, charge to mass ratio and the presence of iduronic and/or glucuronic acid content as well as the relationships between the mono- and disaccharide building blocks, and active sites associated with these building blocks. As described herein, it is possible to use specific chemical signatures to formulate polysaccharides with one or more reduced therapeutic activity and/or enhanced delivery properties. It is also possible to use specific chemical signatures to formulate polysaccharides that demonstrate decreased permeability of epithelial tissue, e.g., as compared to a reference standard. The chemical signature can be provided by determining one or more primary outputs chosen from the following: the presence or the amount of one or more component saccharides or disaccharides; the presence or the amount of one or more block components, wherein a block component is one made up of more than one saccharides or polysaccharide;

The presence or amount of one or more saccharide-representative, wherein a saccharide-representative is a saccharride modified to enhance detectability; the presence or amount of an indicator of three dimensional structure or a parameter related to three dimensional structure, e.g., activity, e.g., the presence or amount of a structure produced by cross-linking a polysaccharide, e.g., the cross-linking of specific saccharides which are not adjacent in the linear sequence; or the presence or amount of one or more modified saccharides, wherein a modified saccharide is one present in a starting material used to make a preparation but which is altered in the production of the preparation, e.g., a saccharide modified by cleavage. The chemical signature can also be provided by determining a secondary output, which include one or more of: total charge; density of charge.

The nomenclature “ΔU” refers to an unsaturated Uronic acid (Iduronic acid (I) or Glucuronic acid (G) that has a double bond introduced at the 4-5 position as a result of the lyase action of heparinases. Upon the introduction of the double bond the distinction between the stereo isomers I and U disappears, and hence the notation ΔU: Δ to denote double bond, and U to denote that they can be derived from either I or U. Thus, as used herein, “ΔU” represents both I and G, such that ΔU_{2S}H_{NS,6S} encompasses both I_{2S}H_{NS,6S} and G_{2S}H_{NS,6S}; ΔU_{2S}H_{NS} encompasses both I_{2S}H_{NS} and G_{2S}H_{NS}, and so forth.

The process of identifying chemical properties or signatures of a polysaccharide and using this information to generate polysaccharides with one or more reduced therapeutic activity and/or enhanced in vivo delivery capabilities or decreased transport across epithelial barriers is referred to herein as the process of chemical formulation of a polysaccharide. For example, this information can be used to generate information about structures in heparins that play a role in anti-Xa activity, anti-IIa activity, or other activities of heparins and to use this information to reduce one or more of these activities of heparin.

Chemical formulation involves the preparation of a composition using chemical entities to achieve an appropriate balance for delivery of an active agent, e.g., while reducing the therapeutic activities associated with the particular polysaccharide. The chemical formulation is accomplished using techniques to structurally characterize or sequence polysaccharides and then formulating, e.g., modifying one or more monosaccharide and/or modifying a linkage or a substituent of that monosaccharide such as masking charge or adding a charge based on the structure. This is distinct from physical formulation of a polysaccharide, which refers to the processing of a particle by methods known in the art based on the physical attributes of the particle such as particle size, tap density, etc. that are all physical descriptions of particles. The compositions and methods described herein include chemical formulation of polysaccharides for efficient delivery of an active agent with reduced side effects of the polysaccharide and chemical formulation of polysaccharides to decrease delivery of an agent, e.g., an agent used in biological warfare, across epithelial barriers. In addition to the chemical formulation, the polysaccharides may be physically formulated to achieve, e.g., a particular particle size, tap density etc. It has been found that some chemical formulations can enhance pulmonary delivery of an active agent without being physically formulated. One specific chemical property that may be analyzed is charge. Neutralization of the charge of a polysaccharide can, e.g., enhance the ability the polysaccharide to permeate lipid membranes, or permeate epithelial barriers. As used herein the terms “neutralization”, “neutralize” and “neutralizing” refer a process for generating a polysaccharide in which the net negative or positive charge of the material has been reduced or masked by at least 10% and in some embodiments by at least 20%, 30%, 40%, 50%, 60%, 70%, 80, 90% or 100 or any integer in between. The net or overall charge of a polysaccharide such as heparin can be calculated by dividing the mass of the heparin by the average molecular weight of a disaccharide (500) and multiplying that number by the average charge per disaccharide (e.g., 2.3). The average charge per disaccharide can vary from polysaccharide to polysaccharide. The average charge is the mean charge for the polysaccharides present in a polydisperse composition. The net charge of each polysaccharide in a composition can vary. Methods of determining the charge of polysaccharides including the charge per disaccharide are described, for example, in Venkataraman, G. et al. Science, 286, 537-542 (1999). Charge neutralization may be accomplished in a variety of ways. Preferably, the charge of the polysaccharide is determined. Based on that determination, an appropriate strategy for charge neutralization may be selected, e.g., a strategy which maintains or enhances the delivery properties of the polysaccharide. In general, a more highly charged polysaccharide will be more effectively neutralized with the use of a higher concentration of neutralizing agent to mask the charge. For instance, chemical analysis of a heparin oligosaccharide revealed that the molecule contained a total of 17 negative charges, primarily O-sulfates. Charge neutralization and powder formation of the heparin molecule was accomplished by precipitating the polysaccharide using a 200 mM sodium chloride pH 4.5 solution. Similarly, a heterogeneous population of heparin, such as a low molecular weight heparin was chemically analyzed and found to have an average charge distribution of 24-32 negative charges. Charge neutralization and optimal powder formation of this material was accomplished by using a higher concentration of salt, counterions, and/or a different pH to effectively mask charge.

The neutralization may be accomplished using a charge neutralization agent. A “charge neutralization agent” as used herein is a positively or negatively charged compound that is capable of interacting with an oppositely charged molecule and thereby neutralizing the charge. Charge neutralization agents include but are not limited to counter ions such as mono- and divalent ions including, but not limited to, barium, calcium, sodium, potassium, lithium, ammonium, magnesium and zinc as well as transition metals such as iron, nickel, and copper; and other neutralizing compounds such as small organic compounds, spermine, spermidine, low molecular weight protamine, or basic peptides.

If a polysaccharide is negatively charged, a positively charged compound may be used to neutralize the polysaccharide. Likewise, if the polysaccharide is positively charged, then a negatively charged compound may be used. Once the type and quantity of charge in the polysaccharide is determined, e.g., by chemical analysis, then the appropriate amount of neutralizing compound may be selected. The exact amount neutralizing compound will depend on the particular sample, since the type and amount of charge may vary from sample to sample. In general, a low concentration of neutralizing agent will be sufficient to reduce the charge of a polysaccharide having only a few charged moieties and it is desirable to increase the concentration of the neutralizing agent for more highly charged molecules.

Another chemical property of the polysaccharides that may be considered is the quantity of 2-O sulfated iduronic acid moieties present in the polysaccharide. 2-O sulfated iduronic acid moieties chelate metals in a distinctly different matter than other components of a polysaccharide. As such the nature and amount of counter ions useful for neutralization is somewhat determined by the number and localization of 2-O sulfated iduronic acids in the polysaccharide. For instance, a heparin with a high degree of 2-O sulfated iduronic acid (˜80%) was efficiently precipitated using calcium or barium salts instead of sodium salts whereas a heparan sulfate with a low degree of 2-O sulfated iduronic acid was not precipitated in an appropriate manner using these same conditions. In general, a higher degree of 2-O sulfated iduronic acids in a polysaccharide is more effectively formulated with a higher concentration of neutralizing agents.

Additionally, the length of the polysaccharide has an impact on its formulation. Based on the length of the polysaccharide, different types and concentrations of organic modifiers such as organic solvents will have different effects on the formulation properties of the polysaccharide. For instance, different sized heparin oligosaccharides were demonstrated to form optimal powders at various concentrations of organic solvent. In general, the longer an oligosaccharide chain, and the higher its number of charges, the less soluble a polysaccharide is in non-aqueous solutions. As such, based on size and charge density as chemical signatures, powders can be formed via the addition of various volume equivalents of organic modifiers. In general, the longer an oligonucleotide within a particular class of polysaccharides (i.e., HLGAGs), a lower concentration of organic modifier will produce enhanced results.

An organic modifier as used herein is an organic solution such as, for instance, an alcohol and a polar organic solvent, such as acetonitrile, acetone, or dimethylsulfoxide and aqueous mixtures thereof.

The activity, size, level of sulfation and/or charge of a polysaccharide can be reduced by digesting the polysaccharide with at least one agent. The agent can be selected, e.g., based upon the information obtained regarding the chemical signature of the polysaccharide. For example, enzymes and/or chemicals can be used which selectively cleave the polysaccharide. Thus, polysaccharides can be generated such that, e.g., regions of the polysaccharide which are involved and/or influence a biological activity can be cleaved, and regions of the polysaccharide which are not involved and/or do not influence a biological activity remain intact. As used herein, the term “intact” means uncleaved and complete.

For example, a LMWH can be generated which has a reduction in at least one activity, e.g., anti-Xa activity and/or anti-IIa activity. Examples of activities mediated by heparin include: anti-Xa activity, anti-IIa activity, protamine neutralization, anticoagulation/antithrombosis, cell proliferation, e.g., unwanted cell proliferation, e.g., unwanted malignant or non-malignant cell proliferation; angiogenesis; inflammatory processes; cell migration; cell activation; cell adhesion. Standard methods of measuring such activities are known. For example, anti-Xa activity can be measured by the amidolytic method on a chromogenic substrate described by Teien et al., Thrombo. Res. 10:399-410 (1977), with a standard being the first international standard for LMWH. Known methods for measuring anti-IIa activity are described, for example, by Anderson et al., Thrombo. Res. 15:531-541 (1979), with a standard being the first international standard for LMWH.

HLGAG fragments may be degraded using for example, enzymes such as heparin lyase enzymes (heparinases) or nitrous acid. They may also be modified using different enzymes that transfer sulfate groups to the specific positions or remove the sulfate groups from those positions. The modifying enzymes are exolytic and nonprocessive which means that they just act once on the non-reducing end and will let go of the heparin chain without sequentially modifying the rest of the chain. For each of the modifiable positions in the disaccharide unit there exits a modifying enzyme. An enzyme that adds a sulfate group is called a sulfotransferase and an enzyme that removes a sulfate group is called a sulfatase. The modifying enzymes include 2-O sulfatase/ sulfotransferase, 3-O sulfatase/sulfotransferase, 6-O sulfatase/sulfotransferase and N-deacetylase-N-sulfotransferase. The function of these enzymes is evident from their names, for example a 2-0 sulfotransferase transfers a sulfate group to the 2-O position of an iduronic acid (2-O sulfated glucuronic acid is a rare occurrence in the HLGAG chains) and a 2-O sulfatase removes the sulfate group from the 2-O position of an iduronic acid.

HLGAG degrading enzymes include but are not limited to heparinase-I, heparinase-II, heparinase-III, heparinase-IV, heparanase, D-glucuronidase and L-iduronidase, modified versions of heparinases, variants and functionally active fragments thereof. The three heparinases from Flavobacterium heparinum are enzymatic tools that have been used for the generation of LMWH (5,000-8,000 Da) and ultra-low molecular weight heparin (≦3,000 Da). Heparinase I cleaves highly sulfated regions of HLGAGs at 2-O sulfated uronic acids, whereas heparinase II has a broader substrate specificity and cleaves glycosidic linkages containing both 2-O sulfated and nonsulfated uronic acids (Ernst, S., Langer, R., Cooney, C. L. & Sasisekliaran, R. (1995) Crit Rev Biochem Mol Biol 30, 3 87-444). Heparinase III, as opposed to heparinase I, cleaves primarily undersulfated regions of HLGAGs, viz., glycosidic linkages containing a nonsulfated uronic acid (Emst, S., Langer, R., Cooney, C. L. & Sasiseldiaran, R. (1995) Crit Rev Biochem Mol Biol 30, 387-444). Several patents and patent applications describe useful modifications and variants and fragments of heparinase, including U.S. Pat. No. 6,217,863 and pending applications Ser. Nos. 09/384,959 and 09/802,285. Other modifications and variants are also useful.

Glucuronidase and iduronidase, as their name suggests, cleave at the glycosidic linkage after a glucuronic acid and iduronic acid respectively. Nitrous acid clips randomly at glycosidic linkages after a N-sulfated hexosamine and converts the six membered hexosamine ring to a 5-membered anhydromannitol ring.

Chemicals useful for digesting polysaccharides such as HLGAGS include chemicals chosen from group consisting of oxidative depolymerization with H₂O₂ or Cu⁺ and H₂O₂, deaminative cleavage with isoamyl nitrite, or nitrous acid, β-eliminative cleavage with benzyl ester of heparin by alkaline treatment or by heparinase.

Methods for identifying the charge and other properties of polysaccharides have been described in Venkataraman, G., et al., Science, 286, 537-542 (1999), and U.S. patent applications Ser. Nos. 09/557,997 and 09/558,137, both filed on Apr. 24, 2000, which are hereby incorporated by reference.

Formulated Polysaccharide Compositions

It was found that the polysaccharides of the invention can be used to deliver an active agent without additional agents that enhance delivery or slow release and still result in therapeutically effective levels of the active agent being delivered by various routes, e.g., pulmonary routes. Formulations of some of the polysaccharides described herein can also be used to decrease the permeability of various agents.

The compositions can also be generated to be in solid or liquid form. An example of a solid form is dry particles, e.g., dry particles for pulmonary delivery such as those described in PCT Publication Number 02/32406, the contents of which are incorporated herein by reference. In some embodiments, the composition contains polysaccharide particles have a mean aerodynamic diameter about 1 to 50 microns, preferably about 1 to 20 microns, or 1 to 10 microns. Such particles can be in a liquid, aerosol or dry powder formulation.

The polysaccharides of the invention may optionally be formulated in a pharmaceutically acceptable carrier. Such preparations may routinely contain pharmaceutically acceptable concentrations of salt, buffering agents, preservatives, compatible carriers, adjuvants, and optionally other therapeutic ingredients. The compositions may further be formulated into specific delivery devices. As described below, the polysaccharide may also be formulated based upon their intended route of delivery.

The compositions of the invention may be administered per se (neat) or in the form of a pharmaceutically acceptable salt. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, oxalic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include, e.g.: acetic acid and a salt (1-2 mole % W/V); citric acid and a salt (1-3 mole % W/V); boric acid and a salt (0.5-2.5 mole % W/V); and phosphoric acid and a salt (0.8-2 mole % W/V). Suitable preservatives include, e.g., benzalkonium chloride (0.003-0.03 mole % W/V); chlorobutanol (0.3-0.9 mole % W/V); parabens (0.01-0.25 mole % W/V) and thimerosal (0.004-0.02 mole % W/V).

The present invention provides pharmaceutical compositions, for medical use, which comprise a polysaccharide preparation together with one or more therapeutic or prophylactic agents and, optionally, a pharmaceutically acceptable carrier and/or other therapeutic ingredients. The term “pharmaceutically-acceptable carrier” as used herein means one or more compatible solid or liquid filler, dilutants or encapsulating substances which are suitable for administration to a human or other animal. The components of the pharmaceutical compositions also are capable of being commingled with the formulations of the present invention, and with each other, in a manner such that there is no interaction which would substantially impair the desired pharmaceutical efficiency of the therapeutic or prophylactic agent in the formulation.

Controlled release of the active agent can also be achieved with appropriate excipient materials that are biocompatible and biodegradable. These polymeric materials which effect slow release of the active agent may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

It has been found that the polysaccharide of the invention can deliver an effective amount of an active agent regardless of the size of the agent to be delivered. Thus, particles, e.g., particles which include a polysaccharide and an active agent, can be greater than 5, 10, 15, 20, 25, 30 microns and still be administered in vivo in therapeutically effective amounts by certain routes of administration, e.g., pulmonary delivery.

Non-Invasive Routes of Administration

The polysaccharides of the invention can be delivered in vivo by various non-invasive routes of delivery. Non-invasive delivery refers to routes of delivery which do not require forced insertion of the polysaccharide through tissue, e.g., a layer of skin. Examples of non-invasive delivery methods which can be used with the polysaccharides of the invention include pulmonary (e.g., by inhalation or nasal delivery), transdermal, and mucosal delivery (e.g., oral, ocular, buccal, sublingual, rectal or vaginal delivery). Invasive delivery methods, which require, e.g., forced pressure or an instrument to deliver through tissue, include intravenous, intramuscular and subcutaneous delivery.

Non-invasive delivery routes have several benefits including the ease of self administration by a subject, e.g., the polysaccharide composition can be in a dosage unit form. Dosage unit form as used herein refers to physically discrete units suited as unitary dosages for the subject to be treated; each unit containing a predetermined quantity of active compound calculated to produce the desired therapeutic effect. Examples of compositions which can be used for self administration include: metered amounts of a composition to be administered from an inhaler for pulmonary delivery; tablets having a prescribed dosage unit for oral administration; transdermal patches to deliver a dosage unit across the skin; and suppositories to deliver a desired dosage unit rectally or vaginally. The compositions can be included in a container, pack, or dispenser together with instructions for administration. These methods, as well as other methods used for non-invasive delivery, may also be used by health care professionals to administer the polysaccharides of the invention to a subject.

For delivery to the skin, the agent/polysaccharide composition can be in the form of aqueous-based solutions, gels, suspensions, lotions, creams, ointments, patches, and the like.

It is understood that the specific route of administration and dose level will depend upon a variety of factors including the activity of the specific compound employed, the age, body weight, general health, gender, and diet of the subject, the time of administration, the desired rate of absorption, bioavailability, the rate of excretion, any drug combination, and the location of desired therapeutic effect, e.g., local or systemic effect. A local therapeutic effect refers to a biologic effect that occurs at the tissue where the polysaccharide is delivered. Non-invasive routes which can be used to deliver a local therapeutic effect include pulmonary, nasal, ocular, transdermal, buccal, rectal and vaginal delivery. A systemic effect refers to a biologic effect that occurs outside of the tissue/organ where the composition is delivered, e.g., the biological effect occurs in the blood.

Pulmonary Administration

It was found that some of the polysaccharides described herein can provide enhanced formulations for delivering an active agent, e.g., a therapeutic or prophylactic agent, by a pulmonary route, e.g., by inhalation through the mouth or nasal passage. In addition, modification of the polysaccharide, e.g., by neutralizing or enhancing the net charge of a polysaccharide, such as an HLGAG, reducing the size of a polysaccharide, such as an HLGAG, reducing the activity level of a polysaccharide, e.g., an HLGAG, and/or decreasing the level of sulfation, e.g., N-sulfation, of a polysaccharide, such as an HLGAG, can enhance the ability of the active agent to permeate a lipid membrane, e.g., epithelial barriers, of the lung. The term “pulmonary tissue” as used herein refers to any tissue of the respiratory tract and includes both the upper and lower respiratory tract, except where otherwise indicated.

Pulmonary delivery routes have several benefits including the ease of self-administration by a subject, e.g., the polysaccharide/active agent composition can be in a dosage unit form of the active agent. Dosage unit form as used herein refers to physically discrete units suited as unitary dosages for the subject to be treated; each unit containing a predetermined quantity of active agent calculated to produce the desired therapeutic effect. An example of a composition which can be used for self-administration include: metered amounts of a composition to be administered from an inhaler for pulmonary delivery. For example, metered amounts of a polysaccharide/insulin composition can provide therapeutically effective amounts of insulin to the subject having diabetes. In preferred embodiments, the polysaccharide is a heparin, e.g., a LMWH, e.g., ardeparin or enoxaparin, e.g., ardeparin or enoxaprin that has been modified as described herein. The compositions can be included in a container, pack, or dispenser together with instructions for administration. These methods, as well as other methods used for pulmonary delivery, may also be used by health care professionals to administer the polysaccharide/active agent composition to a subject.

It is understood that the dose level will depend upon a variety of factors including the activity of the specific agent employed, the age, body weight, general health, gender, and diet of the subject, the time of administration, the desired rate of absorption, bioavailability, the rate of excretion, any drug combination, and the location of desired therapeutic effect, e.g., local or systemic effect. A local therapeutic effect refers to a biologic effect that occurs at the tissue where the active agent is delivered. For instance, when the active agent is used for treating or preventing a localized reaction in the lung, it may be desirable to deliver the active agent to the lung to produce a local effect for the treatment of, e.g., a respiratory disease or a lung disease. A systemic effect refers to a biologic effect that occurs outside of the respiratory system where the active agent is delivered, e.g., the biological effect occurs after delivery to the blood.

For administration by inhalation, the polysaccharide/active agent composition can be delivered in the form of an aerosol spray from a nebulizer or a pressured container or dispenser which contains a suitable propellant. The polysaccharide/active agent composition be in the form of a dry particle or as a liquid.

The polysaccharide/active agent composition may be conveniently delivered in the form of an aerosol spray presentation from pressurized packs or a nebulizer, with the use of a suitable propellant, e.g., dichlorodifluoromethane, trichlorofluoromethane, dielilorotetrafluoroctliane, carbon dioxide or other suitable gas. In the case of a pressurized aerosol, the dosage unit may be determined by providing a valve to deliver a metered amount of the active agent. Other devices can include those driven by patient administration. Capsules and cartridges for use in an inhaler or insufflator may be formulated containing a powder mix of the polysaccharide, the active agent and a suitable powder base such as lactose or starch, if the particle is a formulated particle. In addition, the polysaccharide/active agent compositions, can be administered with other materials such as 100% DPPC or other surfactants can be mixed with the polysaccharide/active agent composition to promote the delivery and dispersion of the active agent. Methods of preparing dry polysaccharide particles are described, for example, in PCT Publication WO 02/32406.

Delivery to the nose can be, e.g., in the form of solutions, gels, or suspensions. The nasal formulations may be formulated, for example, into an aqueous or partially aqueous solution, which can then be utilized in the form of a nasal drop or an aerosol.

The polysaccharide/active agent composition when administered by pulmonary routes can result in the active agent being rapidly absorbed, thereby producing a rapid local or systemic therapeutic result. It has been discovered the polysaccharide can deliver an active agent such that the peak activity of the delivered active agent can be achieved within 3 to 4 hours, and preferably within two hours, after delivery. In some embodiments, the peak activity of the active agent can be achieved even more quickly, e.g., within one half hour or even within ten minutes. In some embodiments, the polysaccharide/active agent composition can be formulated for longer biological half-life of the active agent

In one embodiment, the polysaccharide is delivered in an amount such that at least 1%, 5%, 10%, 15%, 20% or more of the polysaccharide/active agent composition is delivered to the upper lung and/or lower lung. In other embodiments, at least 20%, 30%, 40%, 50%, 60%, 70%, or 80% of the polysaccharide/active agent composition is delivered to the upper lung and/or lower lung. In one embodiment, the polysaccharide/active agent composition is provided in a metered dose using, e.g., an inhaler or nebulizer. Preferably, the active agent is delivered in a dosage unit form of at least about 0.001 mg of active agent/puff, 0.5 mg of active agent/puff, 1 mg of active agent/puff, 2 mg of active agent/puff, 5 mg of active agent/puff, 10 mg of active agent/puff, 15 mg of active agent/puff, 20 mg of active agent/puff, 25 mg of active agent/puff, 30 mg of active agent/puff, 35 mg of active agent/puff, 40 mg of active agent/puff, 45 mg of active agent/puff, 50 mg of active agent/puff, 55 mg of active agent/puff, 60 mg of active agent/puff, 70 mg of active agent/puff, 80 mg of active agent/puff, 90 mg of active agent/puff, 100 mg of active agent/puff or more.

The percent bioavailability can be calculated as follows: the percent bioavailability=(AUCₙₒₙ₋ᵢₙᵥₐₛᵢᵥₑ/AUC_{i.v. or s.c.})×(dose_{i.v. or s.c.}/doseₙₒₙ₋ᵢₙᵥₐₛᵢᵥₑ)×100.

Although not necessary to achieve the desired levels of delivery, delivery facilitators such as surfactants can be used to enhance pulmonary delivery. A “surfactant” as used herein refers to a compound having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; phospholipids.

Mucosal Delivery

It was found that various of chemical properties of polysaccharides, e.g., activity level, size, charge and/or level of sulfation can be used to generate enhanced compositions for mucosal delivery of compounds. The terms “mucosa” and “mucosal” refer to mucous tissue, epithelium, lamina propria and the layer of smooth muscle in the digestive and reproductive tract. Methods of mucosal delivery include ocular, oral, buccal, sublingual, rectal and vaginal delivery.

For oral administration, the active agent/polysaccharide composition can be formulated by combining the active compound(s) with a pharmaceutically acceptable carrier. Such carriers allow the compounds of the invention to be formulated as tablets, pills, dragees, capsules, liquids, gels, syrups, slurries, suspensions and the like, for oral ingestion by the subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients include fillers such as sugars, (e.g., lactose, sucrose, mannitol or sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP)). If desired, disintegrating agents may be added, such as cross-linked polyvinylpyrrolidone, agar or alginate may also be formulated in saline buffers for neutralizing internal acid conditions or may be administered without any carriers.

Dragee cores are provided with suitable coatings. For this purposes, concentrated sugar solutions can be used, which may optionally contain gum arabic, talc, polyvinylpyrrolidine, carbopol gell, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain active ingredients in admixture with fillers such as lactose, binders such as starches, and/or lubricants such as talc or magnesium stearate and, optionally stabilizers. In soft capsules, the active compounds may be dissolved or suspended in suitable liquids such as fatty oils, liquid paraffin, or liquid polyethylene glycols. In addition, stabilizers may be added. Microspheres formulated for oral administration may also be used. Such microspheres are known in the art. It is advantageous to formulate oral compositions in appropriate dosage units.

When a polysaccharide composition of the invention is being delivered orally such that the polysaccharide must pass through a membrane, e.g., the intestinal mucosa, to achieve systemic delivery, delivery enhancers such as penetrants appropriate to the barrier to be permeated can be used in the formulation. Such penetrants are generally known in the art, and include, for example, detergents, bile salts, and fusidic acid derivatives.

Transmucosal administration can be accomplished through rectal delivery by, e.g., the use of suppositories (e.g., with conventional suppository bases such as cocoa butter and other glycerides) or retention enemas for rectal delivery.

For buccal administration, the compositions may take the form of tablets, lozenges and mouth rinses formulated in conventional manner.

The term “therapeutically acceptable levels” refers to the delivery of an active agent/polysaccharide composition, for local or systemic effect, at a level sufficient to result in the occurrence of a desired activity in a cell or subject. A “therapeutically effective amount” refers to an amount of the active agent/polysaccharide composition which is effective, upon single or multiple dose administration to a subject, in treating, alleviating, relieving or improving a symptom of a subject as described herein beyond that expected in the absence of such treatment.

Ocular Delivery

The formulations and methods described herein can be used, e.g., to enhance delivery of an agent to the eye. Such formulations and methods can be used, e.g., to treat various disorders affecting the eye including, but not limited to, various ocular neovasculature disorders (e.g., macular degeneration and Stargardt disease); macular edema, posterior uveitis, retinosa pigmentosa, diabetic retinopathy, Usher syndrome, and various viral infections.

Stargardt disease is the most common form of inherited juvenile macular degeneration. It is characterized by a reduction of central vision with a preservation of peripheral (side) vision. Macular degeneration is a retinal degenerative disease that causes progressive loss of central vision. The risk of developing macular degeneration increases with age. The disease most often affects people in their sixties and seventies. Macular degeneration is the most common cause of vision loss in individuals over the age of fifty-five. Retinitis pigmentosa (RP) is the name given to a group of inherited eye diseases that affect the retina. Retinitis pigmentosa causes the degeneration of photoreceptor cells in the retina. Photoreceptor cells capture and process light helping us to see. As these cells degenerate and die, patients experience progressive vision loss. Glaucoma is a progressive disease which leads to optic nerve damage and, ultimately, total loss of vision. The causes of this disease have been the subject of extensive studies for many years, but are still not fully understood. The principal symptom of and/or risk factor for the disease is elevated intraocular pressure or ocular hypertension due to excess aqueous humor in the anterior chamber of the eye.

Various agents that can be delivered to the eye, e.g., by topical delivery, using the methods and formulations described herein include: antibiotic agents such as fumagillin analogs, minocycline, fluoroquinolone, cephalosporin antibiotics, herbimycon A, tetracycline, chlortetracycline, bacitracin, neomycin, polymyxin, gramicidin, oxytetracycline, chloramphenicol, gentamicin and erythromycin; antibacterial agents such as sulfonamides, sulfacetamide, sulfamethizole, sulfoxazole, nitrofurazone, and sodium propionate; antiviral agents such as idoxuridine, famvir, trisodium phosphonoformate, trifluorothymidine, acyclovir, ganciclovir, DDI and AZT, protease and integrase inhibitors; anti-glaucoma agents such as beta blockers (timolol, betaxolol, atenolol), prostaglandin analogues, hypotensive lipids, and carbonic anhydrase inhibitors, miotics (e.g., pilocarpine), sympathomimetics, macrophage derived factors and/or neutrotrophic factors (e.g., TNF-beta, oncomodulin); antiallergenic agents such as antazoline, methapyriline, chlorpheniramine, pyrilamine and prophenpyridamine; antiinflammatory agents such as hydrocortisone, leflunomide, dexamethasone phosphate, fluocinolone acetonide, medrysone, methylprednisolone, prednisolone phosphate, prednisolone acetate, fluoromethalone, betamethasone, triamcinolone acetonide, adrenalcortical steroids and their synthetic analogues, and 6-mannose phosphate; antifungal agents such as fluconazole, amphotericin B, liposomal amphotericin B, voriconazole, imidazole-based antifungals, triazole antifungals, echinocandin-like lipopeptide antibiotics, lipid formulations of antifungals; polycations and polyanions such as suramine and protamine; decongestants such as phenylephrine, naphazoline, and tetrahydrazoline; anti-angiogenesis compounds including those that can be potential anti-choroidal neovascularization agents such as 2-methoxyestradiol and its analogues (e.g., 2-propynl-estradiol, 2-propenyl-estradiol, 2-ethoxy-6-oxime-estradiol, 2-hydroxyestrone, 4-methoxyestradiol), VEGF antagonists such as VEGF antibodies and VEGF antisense, angiostatic steroids (e.g., anecortave acetate and its analogues, 17-ethynylestradiol, norethynodrel, medroxyprogesterone, mestranol, androgens with angiostatic activity such as ethisterone); adrenocortical steroids and their synthetic analogues including fluocinolone acetonide and triamcinolone acetonide and all angiostatic steroids; immunological response modifying agents such as cyclosporine A, Prograf (tacrolimus), macrolide immunosuppressants, mycophenolate mofetil, rapamycin, and muramyl dipeptide, and vaccines; anti-cancer agents such as 5-fluoroucil, platinum coordination complexes such as cisplatin and carboplatin, adriamycin, antimetabolites such as methotrexate, anthracycline antibiotics, antimitotic drugs such as paclitaxel and docetaxel, epipdophylltoxins such as etoposide, nitrosoureas including carmustine, alkylating agents including cyclophosphamide; arsenic trioxide; anastrozole; tamoxifen citrate; triptorelin pamoate; gemtuzumab ozogamicin; irinotecan hydrochloride; leuprolide acetate; bexarotene; exemestrane; epirubicin hydrochloride; ondansetron; temozolomide; topoteanhydrochloride; tamoxifen citrate; irinotecan hydrochlorise; trastuzumab; valrubicin; gemcitabine HCL; goserelin acetate; capecitabine; aldesleukin; rituximab; oprelvekin; interferon alfa-2a; letrozole; toremifene citrate; mitoxantrone hydrochloride; irinotecan HCL; topotecan HCL; etoposide phosphate; gemcitabine HCL; and amifostine; antisense agents; antimycotic agents; miotic and anticholinesterase agents such as pilocarpine, eserine salicylate, carbachol, diisopropyl fluorophosphate, phospholine iodine, and demecarium bromide; mydriatic agents such as atropine sulfate, cyclopentane, homatropine, scopolamine, tropicamide, eucatropine, and hydroxyamphetamine; differentiation modulator agents; sympathomimetic agents such as epinephrine; anesthetic agents such as lidocaine and benzodiazepam; vasoconstrictive agents; vasodilatory agents; polypeptides and protein agents such as angiostatin, endostatin, matrix metalloproteinase inhibitors, platelet factor 4, interferon-gamma, insulin, growth hormones, insulin related growth factor, heat shock proteins, humanized anti-IL-2 receptor mAb (Daclizumab), etanercept, mono and polyclonal antibodies, cytokines, antibody to cytokines; neuroprotective agents such as calcium channel antagonists including nimodipine and diltiazem, neuroimmunophilin ligands, neurotropins, memantine and other NMDA antagonists, acetylcholinesterase inhibitors, estradiol and ananlogues, vitamin B12 analogues, alpha-tocopherol, NOS inhibitors, antioxidants (e.g. glutathione, superoxide dismutase), metals like cobalt and copper, neurotrophic receptors (Akt kinase), growth factors, nicotinamide (vitamin B3), alpha-tocopherol (vitamin E), succinic acid, dihydroxylipoic, acid, fusidic acid; cell transport/mobility impending agents such as colchicine, vincristine, cytochalasin B; carbonic anhydrase inhibitor agents; integrin antagonists; and lubricating agents, singly or in combinations thereof.

In one embodiment, the formulation is for macular degeneration and the agent is selected from: pegaptanib sodium, ranibizumab, verteporfin and siRNA drugs.

Delivery to the eye can be in the form of solutions, gels, or suspensions. The vehicles will generally be aqueous in nature. Aqueous solutions are generally preferred based on ease of formulation as well as a patient's ability to easily administer such compositions by means of instilling one to two drops of the solutions in the affected eyes. However, other types of compositions, such as suspensions, viscous or semi-viscous gels, or other types of solid or semi-solid compositions can be used.

The ophthalmic compositions of the present invention may also include various other ingredients, such as buffers, preservatives, co-solvents, and viscosity building agents. Ophthalmic products for topical use may be packaged in multidose form. Preservatives can be included to reduce or inhibit microbial contamination. Suitable preservatives include: benzalkonium chloride, thimerosal, chlorobutanol, methyl paraben, propyl paraben, phenylethyl alcohol, edetate disodium, sorbic acid, polyquatemium-1, or other agents known to those skilled in the art. Such preservatives are typically employed at a level of from 0.001 to 1.0% weight/volume (“% w/v”). Such preparations may be packaged in dropper bottles or tubes suitable for safe administration to the eye, along with instructions for use.

Methods for Monitoring Non-Invasive Delivery

The amount of agent delivered can be determined using routine methods. For instance to determine delivery by inhalation, in a test system, lavage of animal lungs at indicated time intervals after inhalation can be used to determine the amount of agent delivered to the lower respiratory tract. Similar tests can be done to determine levels of an agent in, e.g., the intestinal mucosa, at various points after oral delivery. This data can be correlated to that amount which would occur in humans or animals being treated. Alternatively, a label, such as a radioactive or fluorescent label can be attached to the agent and/or polysaccharide and used to determine the distribution of the delivered agent and/or polysaccharide. The amount of agent and/or polysaccharide delivered to a desired tissue can also be determined as the amount of therapeutic effect resulting from the presence of the agent and/or polysaccharide in that tissue or in the region where the biological activity is occurring, e.g., the blood, or the blood plasma concentration of the polysaccharide. The type of parameter used to assess the effectiveness of the delivery will vary depending on a variety of factors including the type of subject, the type of equipment available, and the disorder being treated or prevented. The peak plasma concentration of an agent and/or polysaccharide can be determined by measuring the level of the agent and/or polysaccharide present in the blood over time and determining when the peak level of concentration is reached. The amount of a therapeutic effect or a peak plasma activity can be identified using routine assays. The type of these effects will depend on the therapeutic parameter being assessed.

Kits

Also within the scope of the invention are kits including a polysaccharide described herein along with instructions on how to use the polysaccharide. In some embodiments, the instructions include information on formulating a polysaccharide of the invention with an active agent. In other embodiments, the kit includes a formulation that includes a polysaccharide and an active agent (e.g., as described herein), and the instructions include information for using the formulation to treat, prevent or detect a disorder described herein. In some embodiments, the kit can include one or more other elements including: instructions for use; other reagents, e.g., a label, a therapeutic agent; devices or other materials for preparing the formulation for administration; pharmaceutically acceptable; devices or other materials for administration to a subject; and devices or other materials for monitoring the active agent. The instructions can include instructions for therapeutic application including suggested dosages and/or modes of administration, e.g., in a patient with a disorder described herein. Other instructions can include instructions on coupling of the polysaccharide to an active agent. As discussed above, the kit can include an active agent, e.g., a therapeutic or prophylactic agent, e.g., any of the active agents described herein.

Therapeutic Uses

The compositions and formulations of the invention can be administered to a subject. As used herein, a subject is a vertebrate such as a human, non-human primate, cow, horse, pig, sheep, goat, dog, cat, rabbit, or rodent. The subject can be, e.g., an experimental animal, a veterinary animal, or a human subject.

The pharmaceutical compositions of the invention may include a “therapeutically effective amount” or a “prophylactically effective amount” of an active agent. A “therapeutically effective amount” refers to an amount effective, at dosages and for periods of time necessary, to achieve the desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects. A “therapeutically effective dosage” preferably inhibits a measurable parameter, relative to untreated subjects. The ability of a compound to inhibit a measurable parameter can be evaluated in an animal model system predictive of efficacy in human. Alternatively, this property of a composition can be evaluated by examining the ability of the active agent to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The polysaccharide/active agent includes an active agent other than the polysaccharide that modulates, e.g., increases, movement of the agent across an epithelium movement. These include, for instance, but are not limited to, active agents such as proteins, nucleic acids, small organic or inorganic molecules, that do not have slow release properties, preservatives, etc. Examples of small molecules include, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. An active agent as used herein is any compound which has a diagnostic, prophylactic, or therapeutic effect in a biological organism. The active agents may optionally be proteins, peptides, antibodies, polysaccharides, nucleic acids (e.g., RNA, DNA, PNA, multiplexes of them (e.g.: triplex)), saccharides, glycoproteins, amino acids, viruses, heterogeneous mixtures of macromolecules (e.g., a natural product extract) and hybrid macromolecules (e.g., protein/nucleic acid hybrids, albumin conjugated proteins, drugs with linker inorganic molecules, organic molecules, lipids, glycolipids, or combinations thereof.

A bioactive agent is any compound which has a prophylactic or therapeutic effect in a biological organism. In some embodiments the bioactive agent is any of the drugs described above or one or more of the following agents: adrenergic agent; adrenocortical steroid; adrenocortical suppressant; agents for treating cognition, antiplatelets, aldosterone antagonist; amino acid; anabolic; analeptic; analgesic; anesthetic; anorectic; anti-acne agent; anti-adrenergic; anti-allergic; anti-Alzheimer's, anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; anxiolytics, appetite suppressant; blood glucose regulator; bone resorption inhibitor; bronchodilator; cardiovascular agent; cholinergic; COX1 inhibitors, COX2 inhibitors, direct thrombin inhibitors, depressant; diagnostic aid; diuretic; dopaminergic agent; estrogen receptor agonist; fibrinolytic; fluorescent agent; free oxygen radical scavenger; gastrointestinal motility effector; glucocorticoid; GPIIbIIIa antagonists, hair growth stimulant; hemostatic; histamine H2 receptor antagonists; hormone; human growth hormone, hypocholesterolemic; hypoglycemic; hypolipidemic; hypnotics, hypotensive; imaging agent; immunological agents such as immunizing agents, immunomodulators, immunoregulators, immunostimulants, and immunosuppressants; cytokines, e.g., interferons; insulin; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; proton pump inhibitors, psychotropic; radioactive agent; scabicide; sclerosing agent; sedative; sedative-hypnotic; selective adenosine Al antagonist; serotonin antagonist; serotonin inhibitor; serotonin receptor antagonist; statins, steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor. In preferred embodiments, the active agent is a polypeptide having a molecular weigh of about 5 to 10 kD, 20 to 40 kD, 60 to 80 kD, 100 to 150 kD or more.

The formulations and compositions can include, e.g., an interferon as the active agent. The indications for interferon treatment can include the relapsing forms of multiple sclerosis. Other indications include: a cancer (e.g., cancer of the kidney, melanoma, multiple myeloma, carcinoid tumors, lymphoma and leukemia), or hepatitis (e.g., hepatitis B and hepatitis C).

In some embodiments, the active agent is insulin and the disorder being treated is diabetes. The term “diabetes” or “diabetes mellitus” is intended to have its medical meaning, namely, a metabolic disorder of multiple etiology characterized by chronic hyperglycemia with disturbances of carbohydrate, fat and protein metabolism resulting from defects in insulin secretion, insulin action, or both. Symptoms of Type 1 diabetes include polyuria, polydipsia, blurring of vision and unexplained weight loss. Symptoms of Type 2 diabetes include hyperglycemia, hyperinsulinemia and obesity. A diagnosis of diabetes is often made when any three of these tests is positive, followed by a second positive test on a different day:
Fasting plasma glucose of greater than or equal to 126 mg/dl with symptoms of diabetes.
Casual plasma glucose (taken at any time of the day) of greater than or equal to 200 mg/dl with the symptoms of diabetes.
Oral glucose tolerance test (OGTT) value of greater than or equal to 200 mg/dl measured at a two-hour interval. The OGTT is given over a three-hour time span.

The effects of diabetes mellitus include long-term damage, dysfunction and failure of various organs. In its most severe forms, ketoacidosis or a non-ketotic hyperosmolar state may develop and lead to stupor, coma and, in absence of effective treatment, death. Often symptoms are not severe, or may be absent, and consequently hyperglycemia sufficient to cause pathological and functional changes may be present for a long time before the diagnosis is made. The long-term effects of diabetes mellitus include progressive development of the specific complications of retinopathy with potential blindness, nephropathy that may lead to renal failure, and/or neuropathy with risk of foot ulcers, amputation, Charcot joints, and features of autonomic dysfunction, including sexual dysfunction. People with diabetes are at increased risk of cardiovascular, peripheral vascular and cerebrovascular disease.

Several pathogenetic processes are involved in the development of diabetes. The abnormalities of carbohydrate, fat and protein metabolism are due to deficient action of insulin on target tissues resulting from insensitivity or lack of insulin. Pathological indications of Type I diabetes include a reduction in number and/or size of pancreatic islet β-cells and high presence of lymphatic infiltrates in an around the islets. These lead to consequent insulin deficiency and glucose intolerance. The pathology of Type 2 diabetes includes fibrotic and/or amylin deposits in the islets of the pancreas, and/or a reduction in the size or number of pancreatic islet β-cells.

The methods of the invention include administering, e.g., by pulmonary delivery, a formulation that includes a polysaccharide described herein and insulin to a subject having diabetes such that insulin is efficacious for its intended use. In one embodiment, the bioavailability of the insulin is at least about 10 to 100,000 μIU/ml over a period of about 5 minutes to 5 hours, preferably in a period of less than 1 to 2 hours after delivery. Such methods can include, e.g., delivering a metered dose of the formulation such that each dose includes 0.1 IU/kg insulin, 0.5 IU/kg insulin, 1 IU/kg insulin, 5 IU/kg insulin, 10 IU/kg insulin, 20 IU/kg insulin, 30 IU/kg insulin, 50 IU/kg insulin, 75 IU/kg insulin, 100 IU/kg insulin, 150 IU/kg insulin, 200 IU/kg insulin, 250 IU/kg insulin, 300 IU/kg insulin, and integers in between.

In some embodiments, the active agent is human growth hormone (i.e., Somatotropin). There are several indications for growth hormone treatment, including GHD, cardiovascular risk associated with GHD, pediatric growth failure and Turner's syndrome, and adult HGH deficiency due to pituitary disease, hypothalamic disease, surgery, trauma, radiation therapy, chronic renal insufficiency, Prader-Willi syndrome or growth retardation in children with GHD. In other embodiments, patients include adults who had inadequate growth hormone as children and subsequently identified as growth hormone deficient. In other embodiments, patients include those suffering from AIDS wasting and/or chemotherapy. Other disorders that can be treated or prevented with a polysaccharide/human growth hormone formulation include: pituitary disease (e.g., pituitary tumor, pituitary surgical damage, hypothalmic disease, irradiation or trauma to the pituitary); fatigue syndromes; fibromyalgia; and obesity. Pituitary hypothalmic diseases include subjects with Sheehan's syndrome, autoimmune hypophysitis, or hypophysitis associated with inflammatory conditions such as sarcoidosis.

Patients having GHD have reduced or absent levels of human growth hormone and IGF-I. In growth hormone deficient adults, the effect of the fatty tissue in the absence of growth hormone is increased body fat. The increase in body fat and the absence of IGF-I can produce insulin resistance. The lack of growth hormone and IGF-I in muscle and bone can also result in decreased muscle mass and bone density. The absence of growth hormone and IGF-I can also lead to increased risk of cardiovascular disorders, sometime resulting in death. Various test are available for diagnosing GHD including insulin tolerance tests, and tests utilizing arginine and the hypothalamic releasing hormone for growth hormone, namely GHRH. Such tests are described in the “American Association of Clinical Endocrinologists Medical Guidelines fro Clinical Practice for Growth Hormone Use in Adults and Children—2003 Update”, Endocrinology Practice 9(1):64-76. GHD treatment can be monitored by one or more of the following: increased human growth hormone levels; increased IGF-I levels; increased bone density; increased lean tissue; decreasing adipose tissue; increased cardiac contractility; and enhanced exercise capability.

The polysaccharide/human growth hormone formulations described herein can also be used to treat Turner's syndrome. Turner's syndrome is a disorder affecting girls that is caused by abnormalities of or the absence of an X chromosome. It is frequently associated with short stature. Other symptoms include: shortness of the neck, webbing of the neck, cubitus valgus, shortness of the fourth or fifth metacarpels and metatarsals, a shield shaped chest, and primary hypogonadism.

In some embodiments, the active agent is EPO. Indications for EPO include, for example, anaemia, which can be a disease in its own right or a symptom of another disease.

In other embodiments, a polysaccharide can be chosen for pulmonary delivery that decreases systemic delivery of the agent as compared to pulmonary delivery of the agent in the absence of the polysaccharide. Such formulations can be used, e.g., for local delivery of an active agent, e.g., a therapeutic or prophylactic agent to the pulmonary tissue. These formulations can be valuable, e.g., in treatment of respiratory diseases such as cystic fibrosis, asthma, allergy, emphysema, adult respiratory distress syndrome (ARDS), lung reperfusion injury, idiopathic pulmonary fibrosis, and asbestos-related fibrosis (e.g., black or brown lung).

Cystic fibrosis is a chronic progressive disease affecting the respiratory system. One serious consequence of cystic fibrosis is Pseudomonas aeruginosa lung infection, which by itself accounts for almost 90% of the morbidity and mortality in cystic fibrosis. Therapeutics for treating cystic fibrosis include antimicrobials for treating the pathogenic infection. The formulations described herein can be used to deliver such antimicrobials or other agents useful for treating cystic fibrosis to the lung of a subject having cystic fibrosis.

Asthma is a chronic lung condition characterized by difficulty in breathing. In general, subjects with asthma have extra sensitive or hyperresponsive airways. The airways react by narrowing or obstructing when they become irritated, which creates difficulty for movement of the air in and out of the lungs. This narrowing or obstruction is caused by one or more of airway inflammation (meaning that the airways in the lungs become red, swollen and narrow), and bronchoconstriction (meaning that the muscles that encircle the airways tighten or go into spasm). The following symptoms are associated with asthma: wheezing, coughing, shortness of breath, and chest tightness. The formulations described herein can be used to deliver such therapeutic agents useful for treating asthma to the lung of a subject having asthma.

Pulmonary cancers are broadly classified into small cell or non-small cell. Non-small cell cancers are further divided into adenocarcinomas, bronchoalveolar-alveolar, squamous cell and large cell carcinomas. Approximately, 75-85 percent of lung cancers are non-small cell cancers and 15-25 percent are small cell cancers of the lung. About eighty percent of pulmonary cancers are due to tobacco smoke. Symptoms that may indicate the pulmonary cancer has spread include hoarseness of the voice (due to spread of the cancer to nerves which control the vocal cords), difficulty in swallowing, and swelling of the face, arms and neck. Metastatic spread of the cancer outside the lung and chest can occur with any of the lung cancer types, but most commonly with small cell cancers and adenocarcinomas. Headaches, weakness, numbness or paralysis may indicate spread of the cancer to the brain or spinal cord. Bone pain or pain in the abdomen can be symptoms of cancer spread to these areas. The formulations described herein can be used to deliver such therapeutic agents useful for pulmonary cancer to the lung of a subject having pulmonary cancer.

Pulmonary infections include a variety of disorders including tuberculosis, pneumonia, bronchitis, anthrax infection, Pseudomonas aerginosa, etc. The formulations described herein can be used to deliver such therapeutic agents useful for pulmonary infection, for example one or more antibiotics, to the lung of a subject suffering from pulmonary infection.

Polysaccharides described herein that decrease movement of the agent across an epithelium movement of agents can be used, e.g., to reduce or prevent, exposure of a subject to an agent such as a pathogenic molecule. Pathogenic molecules include virions, allergens and bacteria, e.g., virion, allergen or bacteria known to bind/associate heparan sulfate to cause infection. Examples of pathogenic molecules include, but are not limited to: Clostridium perfringens, Clostridium diphtheriae, Clostridium difficile, Vibrio cholerae, Escherichia coli, bacterioides fragilis, Helicobacter pylori, Dermatophagoides pteronyssinus, reovirus, Coxsackievirus, rotavirus, HSV, HPV, RSV, HIV, and AAV.

Such pathogenic molecules can include, e.g., molecules used in biowarfare. The term “biowarfare” as used herein refers to the use of disease-producing microorganisms, toxic biological products, or organic biocides to cause death or injury to humans, animals, or plants. Examples of molecules used in biowarfare include anthrax, ricin, brucellosis, cholera, Congo-Crimean hemorrhagic fever, ebola hemorrhagic fever, Marburg fever, melioidosis, plague, Q fever, rift valley fever, saxitoxin, smallpox, staphylococcal enterotoxin B, tricothecene mycotoxins, tularemia, Venezuelan equine encephalitis and botulinum toxin.

A number of embodiments of the invention have been described. Nevertheless, it will be understood that various modifications may be made without departing from the spirit and scope of the invention. Accordingly, other embodiments are within the scope of the following claims.

What is claimed:

1. A method of modulating, e.g., increasing or decreasing, movement of an agent across an epithelium, comprising:
contacting epithelial tissue with a soluble polysaccharide capable of altering increasing or decreasing the permeability of, intercellular junctions between epithelial cells; and
contacting epithelial tissue with an agent to thereby modulate movement of the agent through the epithelial tissue.

2. The method of claim 1, wherein the polysaccharide comprises a hexasaccharide or larger polysaccharide.

3. The method of claim 1, wherein the agent is contacted with the epithelial tissue while the modulating effect of the polysaccharide is still detectable.

4. The method of claim 1, wherein the polysaccharide is contacted with the epithelial tissue while an effect of the agent is still detectable.

5. The method of claim 1, wherein the agent is contacted with the epithelial tissue simultaneously with the polysaccharide.

6. The method of claim 1, wherein the polysaccharide is a polysaccharide capable of increasing movement of the agent across an epithelium.

7. The method of claim 6, wherein the agent is contacted with the epithelial tissue while the ability of the polysaccharide to increase movement across an epithelium is still present.

8. The method of claim 6, wherein the polysaccharide is contacted with the epithelial tissue while an effect of the agent is still detectable.

9. The method of claim 6, wherein the agent is contacted with the epithelial tissue simultaneously with the polysaccharide.

10. The method of claim 6, wherein the polysaccharide is an HLGAG.

11. The method of claim 10, wherein the HLGAG is a heparin or a low molecular weight heparin (LMWH).

12. The method of claim 1, wherein the polysaccharide is a polysaccharide capable of decreasing movement of the agent across an epithelium.

13. The method of claim 12, wherein the agent is a pathogenic agent selected from a virion, an allergen or a bacteria.

14. The method of claim 13, wherein the agent is contacted with the epithelial tissue while the ability of the polysaccharide to decrease movement across an epithelium is still present.

15. The method of claim 13, wherein the agent is contacted with the epithelial tissue simultaneously with the polysaccharide.

16. The method of claim 1,wherein the polysaccharide is chondroitin sulfate.

17. The method of claim 1 wherein the polysaccharide is dermatan sulfate

18. The method of claim 1 wherein the polysaccharide is a 2-O desulfated LMWH.

19. The method of claim 1, wherein the epithelial cells are pulmonary epithelial cells.

20. The method of claim 1, wherein the epithelial cells are ocular epithelial cells, dermal epithelial cells, or nasal epithelial cells.

21. The method of claim 1, wherein the polysaccharide is capable of modulating movement of the agent across an epithelium upon contact or within 30 minutes after contact with the polysaccharide.

22. The method of claim 1, wherein the polysaccharide is capable of modulating movement of the agent across an epithelium for a period of about 30 minutes to 5 hours.

23. A method of increasing or decreasing movement of an agent across an epithelium, comprising:
contacting epithelial tissue with a soluble polysaccharide capable of modulating one or more of the location, expression level and activity of one or more intercellular junction proteins; and
contacting epithelial tissue with an agent, to thereby modulate movement of the agent through the epithelial tissue.

24. The method of claim 23, wherein the polysaccharide comprises a hexasaccharide or larger polysaccharide.

25. The method of claim 23, wherein the intercellular junction protein is selected from a claudin, a junction associate molecule (JAM), and an occludin, or a zona occludens.

26. The method of claim 23, wherein the polysaccharide is capable of increasing the expression and/or activity of ZO-1.

27. The method of claim 23, wherein the agent is contacted with the epithelial tissue while the modulating effect of the polysaccharide is still detectable.

28. The method of claim 23, wherein the polysaccharide is contacted with the epithelial tissue while an effect of the agent is still detectable.

29. The method of claim 23, wherein the agent is contacted with the epithelial tissue simultaneously with the polysaccharide.

30. The method of claim 23, wherein the polysaccharide is a polysaccharide capable of increasing movement of the agent across an epithelium.

31. The method of claim 30, wherein the polysaccharide increases the expression and/or activity level of one or more intercellular junction proteins.

32. The method of claim 31, wherein the agent is contacted with the epithelial tissue while the ability of the polysaccharide to increase movement across an epithelium is still present.

33. The method of claim 31, wherein the polysaccharide is contacted with the epithelial tissue while an effect of the agent is still detectable.

34. The method of claim 31, wherein the agent is contacted with the epithelial tissue simultaneously with the polysaccharide.

35. The method of claim 23, wherein the polysaccharide is an HLGAG.

36. The method of claim 35, wherein the HLGAG is a heparin or LMWH.

37. The method of claim 23, wherein the polysaccharide is a polysaccharide capable of decreasing movement of the agent across an epithelium.

38. The method of claim 23, wherein the polysaccharide decreases expression and/or activity of one or more intercellular junction proteins.

39. The method of claim 37, wherein the agent is contacted with the epithelial tissue while the ability of the polysaccharide to decrease movement across an epithelium is still present.

40. The method of claim 37, wherein the agent is contacted with the epithelial tissue simultaneously with the polysaccharide.

41. The method of claim 37, wherein the polysaccharide is a chondroitin sulfate, a dermatan sulfate or a 2-O desulfated heparin.

42. The method of claim 23, wherein the epithelial cells are pulmonary epithelial cells.

43. The method of claim 23, wherein the epithelial cells are ocular epithelial cells, dermal epithelial cells, or nasal epithelial cells.

44. The method of claim 23, wherein the polysaccharide is capable of modulating movement of the agent across an epithelium upon contact or within 30 minutes after contact with the polysaccharide.

45. The method of claim 23, wherein the polysaccharide is capable of modulating movement of the agent across an epithelium for a period of about 30 minutes to 5 hours.

46. A method for enhancing the permeability of intercellular junctions in epithelial tissue, comprising:
contacting the epithelial tissue with a polysaccharide, e.g., a soluble polysaccharide, in an amount to increase expression and/or activity of zona occludins-1 (ZO-1), to thereby enhance the permeability of the intercellular junctions.

47. The method of claim 46, wherein the polysaccharide is chondroitin sulfate.

48. The method of claim 46, wherein the polysaccharide is an HLGAG that has no 2-O sulfate groups or a polysaccharide that is less than 40% 2-O-sulfated.

49. The method of claim 48, wherein the HLGAG has been chemically or enzymatically treated to decrease 2-O sulfation of the polysaccharide by at least 20% or more.

50. The method of claim 48, wherein the HLGAG has been enzymatically treated with a 2-O sulfatase.

51. The method of claim 46, wherein the polysaccharide reduces the ability of an agent to penetrate the epithelial tissue.

52. The method of claim 46, wherein the agent is a pathogenic molecule.

53. The method of claim 52, wherein the pathogenic molecule is selected from virions, allergens and bacteria.

54. The method of claim 53, wherein the virion, allergen or bacteria is known to bind/associate heparan sulfate to cause infection.

55. The method of claim 52, wherein the pathogenic molecule is selected from anthrax, ricin, brucellosis, cholera, Congo-Crimean hemorrhagic fever, ebola hemorrhagic fever, Marburg fever, melioidosis, plague, Q fever, rift valley fever, saxitoxin, smallpox, staphylococcal enterotoxin B, tricothecene mycotoxins, tularemia, Venezuelan equine encephalitis and botulinum toxin.

56. The method of claim 52, wherein the pathogenic molecule is selected from the group consisting of: Clostridium perfringens, Clostridium diphtheriae, Clostridium difficile, Vibrio cholerae, Escherichia coli, bacterioides fragilis, Helicobacter pylori, Dermatophagoides pteronyssinus, reovirus, Coxsackievirus, and rotavirus.

57. The method of claim 52, wherein the pathogenic molecule is a virus selected from the group consisting of: HSV, HPV, RSV, HIV, and AAV.

58. A method of non-mucosal delivery of an effective amount of an agent to a subject,
comprising administering to a subject an effective amount of the agent and a soluble polysaccharide, to thereby deliver the agent to the subject.

59. The method of claim 58, wherein the non-mucosal delivery is transdermal delivery.

60. The method of claim 58 or 59, wherein the polysaccharide comprises a hexasaccharide or larger polysaccharide.

61. The method of claim 59, wherein the therapeutic, prophylactic, or diagnostic agent is selected from the group consisting of: a polypeptide, a nucleic acid, a small molecule, a lipid, and a glycolipid.

62. The method of claim 59, wherein the agent is a polypeptide selected from the group consisting of: insulin, proinsulin, human growth hormone, interferon, α-1 proteinase inhibitor, alkaline phosphotase, angiogenin, cystic fibrosis transmembrane conductance regulator, extracellular superoxide dismutase, fibrogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin.

63. The method of claim 59, wherein agent is selected from the group consisting of: parathyroid hormone and derivatives and fragments thereof, erythropoietin, epoetin beta, gene activated erythropoietin, epoetin beta, second generation EPO, epoetin beta, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, domase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/somatropin, Anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, lenograstim, sargramostim, molgramostim, mirimostim, sargramostim, nartograstim, oprelvekin, peptide tyrosin-tyrosin (PYY), apolipoprotein A-IV, leptin, melanocortin, amylin, orexin, adiponectin, and ghrelin.

64. The method of claim 59, wherein the therapeutic or prophylactic agent is a polypeptide having a molecular weight of about 500 Da to 5 kDa, 5 to 10 kDa, 10 to 20 kDa, 20 to 40 kDa, 50 to 100 kDa, or 100 to 150 kDa, or 150 kDa to 300 kDa.

65. The method of claim 64, wherein the polypeptide has a molecular weight of less than 150 kD, less than 100 kDa, or less than 50 kDa.

66. The method of claim 64, wherein the polypeptide has a molecular weight of 0.5-35 kDa.

67. The method of claim 59, wherein the polysaccharide is an HLGAG.

68. The method of claim 67, wherein the HLGAG is heparin or a LMWH.

69. The method of claim 68, wherein the HLGAG is a LMWH selected from enoxaparin, dalteparin, reviparin, tinzaparin, nadroparin, certoparin, ardeparin, M118 and parnaparin.

70. The method of claim 68, wherein the HLGAG is a heparin or LMWH that has been modified to alter one or more of its charge, size, level of sulfation or therapeutic activity.

71. The method of claim 68, wherein the polysaccharide is in a preparation comprising LMWH wherein all or a portion of the polysaccharides in the preparation consist of about two to twenty monosaccharides.

72. The method of claim 71, wherein the chemical signature of one or more polysaccharides in the preparation has been determined and one or more polysaccharide is modified based upon its chemical signature.

73. The method of claim 72, wherein the heparin or LMWH has an anti-Xa activity and/or an anti-IIa activity that is reduced by at least 30% or more as compared to a reference standard wherein the reference standard is the level of anti-Xa activity and/or anti-IIa activity of a commercially available version of the heparin or LMWH or is the level of anti-Xa activity and/or anti-IIa activity of the heparin or LMWH prior to modification.

74. The method of claim 73, wherein the heparin or LMWH is modified at one or more chemical signature of an oligosaccharide of heparin which comprises the structure ΔUH_{NAc,6S}GH_{NS,3S,6S}, ΔUH_{NS,6S}GH_{NS,3S,6}, ΔUH_{NAc,6S}GH_{NS,3S}, and ΔUH_{NS,6S}GH_{NS,3S}, to reduce the anti-Xa activity and/or the anti-IIa activity of the heparin or LMWH.

75. The method of claim 72, wherein the heparin or LMWH comprises a size that is reduced as compared to a reference standard wherein the reference standard is the level of average chain length of a commercially available version of the heparin or LMWH or is the average chain length of the heparin or LMWH prior to modification.

76. The method of claim 72, wherein the heparin or LMWH comprises a charge that has been modified as compared to a reference standard wherein the reference standard is the charge of a commercially available version of the heparin or LMWH or is the charge of the heparin or LMWH prior to modification.

77. The method of claim 72, wherein the heparin or LMWH has been modified to decrease N-sulfation, e.g., the heparin or LMWH has been chemical or enzymatically treated to decrease N-sulfation of the heparin or LMWH, by at least 20% or more.

78. The method of claim 68, wherein the polysaccharide has no N-sulfate groups or the polysaccharide that is less than 40% N-sulfated.

79. The method of claim 68, wherein the polysaccharide is capable of movement of the agent across an epithelium upon administration or within 30 minutes after administration of the polysaccharide.

80. The method of claim 68, wherein the polysaccharide is capable of movement of the agent across an epithelium for a period of about 30 minutes to 5 hours after administration.

81. The method of claim 68, wherein the polysaccharide and the agent are in a composition further comprising a pharmaceutically acceptable carrier and/or a delivery enhancer.

82. A method of ocular delivery of an effective amount of an agent, comprising administering to a subject an effective amount of an agent and a polysaccharide.

83. The method of claim 82, wherein the polysaccharide comprises a hexasaccharide or larger polysaccharide.

84. The method of claim 82, wherein the agent is selected from the group consisting of: a polypeptide, a nucleic acid, a small molecule, a lipid, and a glycolipid.

85. The method of claim 84, wherein the agent is a polypeptide selected from the group consisting of: insulin, proinsulin, human growth hormone, interferon, α-1 proteinase inhibitor, alkaline phosphotase, angiogenin, cystic fibrosis transmembrane conductance regulator, extracellular superoxide dismutase, fibrogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen. activator, antithrombin III, prolactin, and α1-antitrypsin.

86. The method of claim 82, wherein agent is selected from the group consisting of: parathyroid hormone and derivatives and fragments thereof, erythropoietin, epoetin beta, gene activated erythropoietin, epoetin beta, second generation EPO, epoetin beta, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, domase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/somatropin, Anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, lenograstim, sargramostim, molgramostim, mirimostim, sargramostim, nartograstim, oprelvekin, peptide tyrosin-tyrosin (PYY), apolipoprotein A-IV, leptin, melanocortin, amylin, orexin, adiponectin, and ghrelin.

87. The method of claim 82, wherein the agent is a polypeptide having a molecular weight of about 500 Da to 5 kDa, 5 to 10 kDa, 10 to 20 kDa, 20 to 40 kDa, 50 to 100 kDa, or 100 to 150 kDa, or 150 kDa to 300 kDa.

88. The method of claim 87, wherein the polypeptide has a molecular weight of less than 150 kD, less than 100 kDa, or less than 50 kDa.

89. The method of claim 88, wherein the polypeptide has a molecular weight of 0.5-35 kDa.

90. The method of claim 82, wherein the polysaccharide is an HLGAG.

91. The method of claim 90, wherein the HLGAG is heparin or a LMWH.

92. The method of claim 91, wherein the HLGAG is a LMWH selected from enoxaparin, dalteparin, reviparin, tinzaparin, nadroparin, certoparin, ardeparin, M118 and parnaparin.

93. The method of claim 90, wherein the HLGAG is a heparin or LMWH that has been modified to alter one or more of its charge, size, level of sulfation or therapeutic activity.

94. The method of claim 82, wherein the polysaccharide is in a preparation comprising LMWH wherein all or a portion of the polysaccharides in the preparation consist of about two to twenty monosaccharides.

95. The method of claim 94, wherein the chemical signature of one or more polysaccharides in the preparation has been determined and one or more polysaccharide is modified based upon its chemical signature.

96. The method of claim 93, wherein the heparin or LMWH has an anti-Xa activity and/or an anti-IIa activity that is reduced by at least 30% or more as compared to a reference standard wherein the reference standard is the level of anti-Xa activity and/or anti-IIa activity of a commercially available version of the heparin or LMWH or is the level of anti-Xa activity and/or anti-IIa activity of the heparin or LMWH prior to modification.

97. The method of claim 96, wherein the heparin or LMWH is modified at one or more chemical signature of an oligosaccharide of heparin which comprises the structure ΔUH_{NAc,6S}GH_{NS,3S,6S}, ΔUH_{NS,6S}GH_{NS,3S,6S}, ΔUH_{NAc,6S}GH_{NS,3S}, and ΔUH_{NS,6S}GH_{NS,3S}, to reduce the anti-Xa activity and/or the anti-IIa activity of the heparin or LMWH.

98. The method of claim 93, wherein the heparin or LMWH comprises a size that is reduced as compared to a reference standard wherein the reference standard is the level of average chain length of a commercially available version of the heparin or LMWH or is the average chain length of the heparin or LMWH prior to modification.

99. The method of claim 93, wherein the heparin or LMWH comprises a charge that has been modified as compared to a reference standard wherein the reference standard is the charge of a commercially available version of the heparin or LMWH or is the charge of the heparin or LMWH prior to modification.

100. The method of claim 93, wherein the heparin or LMWH has been chemical or enzymatically treated to decrease N-sulfation of the heparin or LMWH, by at least 20% or more.

101. The method of claim 93, wherein the polysaccharide has no N-sulfate groups or the polysaccharide that is less than 40% N-sulfated.

102. A method of increasing the permeability of epithelial tissue, e.g., by decreasing permeability of intercellular junctions in the epithelial tissue, comprising:
contacting the epithelial tissue with a permeability increasing amount of a polysaccharide, e.g., a soluble polysaccharide, that has no N-sulfate groups or a polysaccharide that is less than 40% N-sulfated, to thereby decrease the permeability of the tight junctions.

103. The method of claim 102, wherein the polysaccharide comprises a hexasaccharide or larger polysaccharide.

104. The method of claim 102, wherein the polysaccharide has been chemical or enzymatically treated to decrease N-sulfation of the polysaccharide, by at least 30% or more.

105. The method of claim 104, wherein the polysaccharide has been treated with pyridine and DMSO to decrease N-sulfation.

106. The method of claim 104, wherein O-sulfation of the polysaccharide is not modified.

107. The method of claim 104, wherein the polysaccharide is a heparin-like glycoaminoglycan (HLGAG), or a heparin sulfate proteoglycan (HSPG).

108. The method of claim 104, wherein the polysaccharide is heparin or a fragment thereof.

109. The method of claim 108, wherein the polysaccharide is a low molecular weight heparin (LMWH).

110. The method of claim 109, wherein none of the glucosamines of the LMWH are N-acetylated.

111. The method of claim 109, wherein the LMWH comprises one or more of a 2-O sulfate, a 3-O sulfate and a 6-O sulfate.

112. The method of claim 109, wherein the LMWH has been chemical or enzymatically treated to decrease N-sulfation of the polysaccharide, by at least 30%, or more.

113. The method of claim 104, wherein the polysaccharide is a digest of heparin which increases permeability through epithelial tissue as compared to a reference, e.g., enoxaparin, dalteparin, reviparin, tinzaparin, nadroparin, certoparin, ardeparin, M118 and parnaparin.

114. The method of claim 113, wherein the LMWH has a molecular weight distribution and/or sugar composition of a selected LMWH reference.

115. The method of claim 112, wherein the LMWH has been treated with pyridine and DMSO to decrease N-sulfation.

116. The method of claim 104, further comprising contacting the epithelial tissue with a therapeutic, prophylactic or diagnostic agent.

117. The method of claim 116, wherein the therapeutic, prophylactic, or diagnostic agent is selected from the group consisting of: a polypeptide, a nucleic acid, a small molecule, a lipid, and a glycolipid.

118. The method of claim 117, wherein the agent is a polypeptide selected from the group consisting of: insulin, proinsulin, human growth hormone, interferon, α-1 proteinase inhibitor, alkaline phosphotase, angiogenin, cystic fibrosis transmembrane conductance regulator, extracellular superoxide dismutase, fibrogen, glucocerebrosidase, glutamate decarboxylase, human serum albumin, myelin basic protein, soluble CD4, lactoferrin, lactoglobulin, lysozyme, lactoalbumin, erythropoietin, tissue plasminogen activator, antithrombin III, prolactin, and α1-antitrypsin.

119. The method of claim 117, wherein agent is selected from the group consisting of: parathyroid hormone and derivatives and fragments thereof, erythropoietin, epoetin beta, gene activated erythropoietin, epoetin beta, second generation EPO, epoetin beta, novel erythropoiesis stimulating protein, insulin lispro, insulin (bovine), insulin, insulin aspart, insulin analogue, Calcitonin, Theraccine, becaplermin (recombinant human platelet derived growth factor-BB), trafermin, human growth hormone-releasing factor, BMP-7, PEG aspariginase, domase alpha, alglucerase, agalsidase-beta, domase alpha, agalsidase-alfa, streptokinase, teneteplase, reteplase, alteplase, pamiteplase, Rh factor VIII, Rh FVIIa, Factor IX (Human), Factor IX (complex), HGH, Somatrem/somatropin, Anti-CD33-calicheamicin conjugate, Edrecolomab, rituxumab, daclizumab, trastuzumab, sulesomab, abciximab, infliximab, muromonab-CD3, palivizumab, alemtuzumab, basiliximab, oprelvekin, gemtuzumab ozogamicin, ibritumomab tiuxetan, sulesomab, palivizumab, interleukin-2, celmoleukin (rIL-2), interferon alfacon-1, interferon alpha, interferon alpha+ribavirin, peg interferon alpha-2a, interferon alpha-2b, interferon alpha 3n, interferon beta-1a, interferon beta, interferon beta 1b, interferon gamma, interferon gamma-1b, filgrastim, lenograstim, sargramostim, molgramostim, mirimostim, sargramostim, nartograstim, oprelvekin, peptide tyrosin-tyrosin (PYY), apolipoprotein A-IV, leptin, melanocortin, amylin, orexin, adiponectin, and ghrelin.

120. The method of claim 116, wherein the therapeutic or prophylactic agent is a polypeptide having a molecular weight of about 500 Da to 5 kDa, 5 to 10 kDa, 10 to 20 kDa, 20 to 40 kDa, 50 to 100 kDa, or 100 to 150 kDa, or 150 kDa to 300 kDa.

121. The method of claim 120, wherein the polypeptide has a molecular weight of less than 150 kD, less than 100 kDa, or less than 50 kDa.

122. The method of claim 120, wherein the polypeptide has a molecular weight of 0.5-35 kDa.

123. A method of delivering an effective amount of an agent to a subject, comprising
administering to the subject a polysaccharide, e.g., a soluble polysaccharide, that has no N-sulfate groups or polysaccharide that is less than 40% N-sulfated, and an effective amount of the agent, to thereby deliver the agent to the subject.

124. A formulation for delivery of a therapeutic, prophylactic or diagnostic agent comprising an effective amount of the agent and a polysaccharide, e.g., a soluble polysaccharide, that has no N-sulfate groups or a polysaccharide that is less than 40% N-sulfated.

125. A formulation for affecting movement across an epithelium of an agent comprising a polysaccharide and an agent, wherein the polysaccharide is capable of modulating intercellular junctions between mammalian epithelial cells.

126. A method of evaluating a polysaccharide to determine if the polysaccharide will modulate permeability of epithelial tissue, comprising:
providing an intercellular junction protein, e.g., a protein selected from a claudin, a junction associate molecule (JAM), anoccludin, and zonula occludin-1);
contacting the protein with a polysaccharide; and
determining if the polysaccharide interacts with, e.g., binds to, the protein, wherein interaction of the polysaccharide with the protein is indicative of a polysaccharide that modulates the permeability of epithelial tissue.

127. A method of evaluating a polysaccharide to determine if the polysaccharide will modulate permeability of epithelial tissue, comprising:
administering a detectable agent, e.g., an antibody or ligand, that binds to an intercellular junction protein, e.g., a protein selected from a claudin, a junction associate molecule (JAM), and an occludin (e.g., zona occluin-1) to a subject;
administering a polysaccharide, e.g., a detectably labeled polysaccharide, to the subject;
detecting the detectable agent that binds to the intercellular junction protein and the polysaccharide to determine whether the intercellular junction protein and the polysaccharide are associated with each other, wherein association of the polysaccharide with the protein is indicative of a polysaccharide that modulates the permeability of epithelial tissue.

128. The method of claim 127, wherein the agent that binds to the intercellular junction protein and/or the polysaccharide are directly or indirectly labeled.
